(19) United States
(12) Patent Application Publication
(10) Pub. No.: US 2016/0223565 A1
(43) Pub. Date: Aug. 4, 2016

(54) BK VIRUS SEROLOGY ASSESSMENT OF PROGRESSIVE MULTIFOCAL LEUKOENCEPHALOPATHY (PML) RISK

(71) Applicant: THE JOHNS HOPKINS UNIVERSITY, Baltimore, MD (US)

(72) Inventor: Raphael Paul VISCIDI, Baltimore, MD (US)

(21) Appl. No.: 15/022,375

(22) PCT Filed: Sep. 18, 2014
(86) PCT No.: PCT/US14/56290

§ 371 Date: Mar. 16, 2016

Related U.S. Application Data
(60) Provisional application No. 61/879,660, filed on Sep. 18, 2013.

(51) Int. Cl. G01N 33/68; C07K 16/28

(52) U.S. Cl. CPC G01N 33/6854; C07K 16/2839; G01N 2469/20; G01N 2333/01; G01N 2800/50; C07K 2317/24; C07K 2317/76

(57) ABSTRACT

The present invention provides methods, compositions, kits, etc. related to the assessment of progressive multifocal leukoencephalopathy (PML) risk, based upon detecting the presence, absence and/or relative levels of serum antibody to BK virus and/or other indication of BK virus infection of a subject, in a subject and/or a sample of a subject. In certain embodiments, such assessment can also include detection of the presence or absence of serum antibody to JC virus in a subject and/or a sample of a subject, optionally to improve the predictive power of such risk assessment.

### CROSS REFERENCE TO RELATED APPLICATIONS

This application is related to U.S. Provisional Patent Application Ser. No. 61/879,660, filed Sep. 18, 2013. The entire contents of this patent application are hereby incorporated by reference herein.

### FIELD OF THE INVENTION

The present invention relates to the assessment of progressive multifocal leukoencephalopathy (PML) risk, i.e. assessing the risk of occurrence of PML, in a subject. The invention also relates to methods based on such risk assessment. In certain embodiments, the invention provides a method of stratifying a subject that is a candidate for undergoing α₄-integrin blocking agent and/or VLA-4 blocking agent treatment, or other therapy (optionally, monoclonal antibody therapy) that targets the immune system, for either initiation or avoidance of such treatment based upon the result(s) of such stratification. In related embodiments, the invention provides a method of stratifying a subject undergoing α₄-integrin blocking agent and/or VLA-4 blocking agent treatment, or other therapy (optionally, monoclonal antibody therapy) that targets the immune system, for suspension of this α₄-integrin/VLA-4 blocking agent or other monoclonal antibody treatment. Further provided are a method of administering an α₄-integrin blocking agent and/or VLA-4 blocking agent treatment, or other therapy (optionally, monoclonal antibody therapy) that targets the immune system, so as to avoid the occurrence of PML.

### BACKGROUND OF THE INVENTION

Monoclonal antibodies targeting the immune system are used to treat a variety of autoimmune disorders and cancers. Several of these drugs and most prominently, natalizumab, which targets the α₄β₁-integrin (VLA-4), have been associated with rare cases of progressive multifocal leukoencephalopathy (PML), a fatal brain infection caused by the human polyomavirus, JC virus (Berger, 2010). Other monoclonal antibody therapies which have been associated with rare cases of PML include rituximab, alemtuzumab, and efalizumab. PML also occurs in 3-5% of human immunodeficiency virus (HIV) infected persons and has been diagnosed in patients with underlying malignancies and organ transplants. For clinical management of patients eligible to receive monoclonal antibody drugs, it is essential to identify risk factors for PML.

### SUMMARY OF THE INVENTION

The present invention relates, at least in part, to the discovery that the absence of and/or a decreased level of serum antibody to BK virus relative to a threshold level in a subject, especially one harboring JC virus (as may also be determined via, e.g., assessment of JC virus titer and/or detection of serum antibody to JC virus in the subject), can be predictive of the likelihood of such a subject to develop progressive multifocal leukoencephalopathy (PML). Thus, the present invention relates, at least in part, to the assessment of PML risk, i.e. assessing the risk of occurrence of PML, in a subject. The invention also relates to methods based on such risk assessment. In certain embodiments, the invention provides a method of stratifying a subject that is a candidate for undergoing α₄-integrin blocking agent and/or VLA-4 blocking agent treatment, or other monoclonal antibody therapy that targets the immune system, for either initiation or avoidance of such treatment based upon the result(s) of such stratification. In related embodiments, the invention provides a method of stratifying a subject undergoing α₄-integrin blocking agent and/or VLA-4 blocking agent treatment, or other monoclonal antibody therapy that targets the immune system, for suspension of this α₄-integrin/VLA-4 blocking agent or other monoclonal antibody treatment. Further provided are a method of administering an α₄-integrin blocking agent and/or VLA-4 blocking agent treatment, or other monoclonal antibody therapy that targets the immune system, so as to avoid the occurrence of PML. Compositions and kits for determination of a PML risk in a subject (e.g., in a sample of a subject) are also provided.

In one aspect, the invention provides a method for identifying a subject at reduced risk of developing progressive multifocal leukoencephalopathy (PML) due to human immunodeficiency virus-associated immunosuppression or upon administration of a humanized monoclonal antibody targeting immune function comprising detecting serum antibody to BK virus in said subject, wherein the presence of serum antibody to BK virus or a level of antibody to BK virus above a threshold level in said subject identifies a subject at reduced risk of developing PML.

In one embodiment, the method further involves administering a humanized monoclonal antibody targeting immune function. Optionally, the humanized monoclonal antibody is natalizumab, rituximab, alemtuzumab or efalizumab.

In another aspect, the invention provides a method for assessing the risk of occurrence of PML in a subject by detecting serum antibody to BK virus in a sample from the subject, where the absence of serum antibody to BK virus or level of antibody to BK virus below a threshold level indicates an increased risk of occurrence of PML in the subject.

In one embodiment, detecting the absence of serum antibody to BK virus or level of antibody to BK virus below a threshold level is based upon comparison to the level of serum antibody to BK virus in a control sample. Optionally, the control sample is of individual(s) who developed HIV-associated immunosuppression or who received the monoclonal antibody therapy and were shown not to develop PML.

In another embodiment, the subject is undergoing α4-integrin blocking agent treatment and/or VLA-4 blocking agent treatment.

In certain embodiments, the method also includes detecting serum antibody to JC virus in the subject.

In some embodiments, the subject has HIV.

In one embodiment, a decision tree analysis is performed to predict the risk of PML in the subject. In another embodiment, at least one of the following inputs is assessed to predict the risk of PML in the subject: OD BK genotype I, OD BK genotype IV, OD JCV, CD4+ T cell count, BK genotype IV (MMR-29), BK genotype IV (THK8) and BK4 competitive inhibition assay result(s).

In certain embodiments, PML is predicted in the subject with sensitivity or specificity of at least 80%, at least 85% or at least 90%.

In a further aspect, the invention provides a method of stratifying a subject undergoing α4-integrin blocking agent treatment and/or VLA-4 blocking agent treatment for suspension of the α4-integrin and/or VLA-4 blocking agent treatment by (i) detecting serum antibody to JC virus in a sample from the subject and (ii) detecting serum antibody to BK virus in a sample from the subject, where: (a) presence of serum antibody to JC virus and/or an elevated level of serum antibody to JC virus, relative to a threshold level and (b) absence of serum antibody to BK virus and/or a decreased level of serum antibody to BK virus, relative to a threshold value, indicates that the subject is in need of a suspension of the α4-integrin-blocking agent treatment and/or VLA-4 blocking agent treatment.

In one embodiment, the subject is being treated for an autoimmune disease or disorder. In certain embodiments, the autoimmune disease or disorder is a pathological inflammatory disease, such as MS, Crohn's disease, sarcoidosis, Sjogren's syndrome, Churg-Strauss syndrome or ulcerative colitis. In other embodiments, the autoimmune disorder is Graves' disease, idiopathic thrombocytopenic purpura, Addison's disease, Hashimoto's thyroiditis, systemic lupus erythematosus or an idiopathic inflammatory myopathy such as dermatomyositis, polymyositis or sporadic inclusion body myositis. In related embodiments, the autoimmune disease or disorder is selected from the group consisting of multiple sclerosis, Crohn's disease, systemic lupus erythematosus, rheumatoid arthritis, psoriasis, and an idiopathic inflammatory myopathy. In certain embodiments, the autoimmune disease or disorder is multiple sclerosis or Crohn's disease.

In one embodiment, the α4-integrin blocking agent and/or the VLA-4 blocking agent is an immunoglobulin or a proteinaceous binding molecule with immunoglobulin-like functions. In a related embodiment, the α4-integrin blocking agent and/or VLA-4 blocking agent is natalizumab.

A further aspect of the invention provides a method for treating or preventing multiple sclerosis (MS), Crohn's or other autoimmune condition in a subject involving detection of serum antibody to BK virus in a subject and administration of a humanized monoclonal antibody targeting immune function to the subject, thereby treating or preventing MS, Crohn's or other autoimmune condition in the subject.

In one embodiment, the other autoimmune condition is systemic lupus erythematosus, rheumatoid arthritis, psoriasis or an idiopathic inflammatory myopathy.

Another aspect of the invention provides a method for treating or preventing a neoplastic condition in a subject that involves detecting serum antibody to BK virus in a subject and administering a humanized monoclonal antibody targeting immune function to the subject, thereby treating or preventing the neoplastic condition in the subject.

In one embodiment, the humanized monoclonal antibody is natalizumab, rituximab, alemtuzumab or efalizumab.

In another embodiment, detection of serum antibody to BK virus involves ELISA.

In one embodiment, detecting serum antibody to BK virus involves differentiating between one or more of the following major genotypes of BK virus: I, II, III and/or IV. In a related embodiment, differentiating of major genotypes of BK virus involves discretely identifying serum antibody to BK virus of major genotype(s) I and/or IV, or involves identifying serum antibody to BK virus of only major genotypes I and IV. Optionally, differentiating between one or more major genotypes of BK virus includes identifying serum antibody specific for a genotype of I, II, III and/or IV, using a method that involves competitive inhibition (blocking) of reactivity with soluble antigen of the genotype.

In a further embodiment, detection is performed upon a sample from the subject. Optionally, the sample is a blood sample or a sample of cerebrospinal fluid.

Another aspect of the invention provides an α4-integrin and/or VLA-4 blocking agent for use in the treatment of multiple sclerosis (MS), Crohn's, other autoimmune condition or a neoplastic condition so as to avoid the occurrence of PML, where the use involves administering of the α4-integrin and/or VLA-4 blocking agent to a subject over a period of time, followed by a discontinuation of the administration for a period of time, wherein discontinuation of the administration of the α4-integrin and/or VLA-4 blocking agent is effected after detecting an absence of serum antibody to BK virus and/or decreased level of serum antibody to BK virus relative to a threshold level in the subject.

In one embodiment, absence and/or the level of serum antibody to BK virus is detected in a sample from the subject.

In another embodiment, serum antibody to JC virus is detected in a sample from the subject. Optionally, detection of serum antibody to JC virus is detected using ELISA or other immunoassay.

A further aspect of the invention provides a kit for determining PML risk of a subject that includes an assay for detection of serum antibody to BK virus, and instructions for its use.

In one embodiment, the assay for detection of serum antibody to BK virus involves ELISA.

In certain embodiments, the kit includes an assay for detecting one or more of the following: BK genotype I, BK genotype IV, JCV, CD4+ T cell count, BK genotype IV (MMR-29), BK genotype IV (THK8) or BK4 competitive inhibition.

### DEFINITIONS

Unless defined otherwise, all technical and scientific terms used herein have the meaning commonly understood by a person skilled in the art to which this invention belongs. The following references provide one of skill with a general definition of many of the terms used in this invention: Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991). As used herein, the following terms have the meanings ascribed to them below, unless specified otherwise.

As used herein, the term “humanized monoclonal antibody targeting immune function” refers to a humanized monoclonal antibody that is capable of altering the immune function of a subject, for example, by binding to, e.g., α₄-integrin (as does natalizumab), CD20 (as does rituximab), CD52 (as does alemtuzumab), CD11a (as does efalizumab), or other immune and/or immune-related target.

The term “administering” as used herein, refers to any mode of transferring, delivering, introducing, or transporting matter such as a compound, e.g. a pharmaceutical compound, or other agent such as an antigen, to a subject. Modes of administration include oral administration, topical contact, intravenous, intraperitoneal, intramuscular, intranasal, or subcutaneous administration. Administration “in combination with” further matter such as one or more therapeutic agents includes simultaneous (concurrent) and consecutive administration in any order.

The term “antibody” generally refers to an immunoglobulin, a fragment thereof or a proteinaceous binding molecule with immunoglobulin-like functions.

The word “assay” as used in this document refers to a method, generally known in the art, to analyse a feature, e.g. the presence, formation or the amount of matter, and/or a catalytic activity occurring in a biological specimen. Such matter may be occurring in a living organism or representing a living organism, such as a protein, a nucleic acid, a lipid, a cell, a virus, a saccharide, a polysaccharide, a vitamin or an ion, to name a few examples. The word “assay” emphasizes that a certain procedure or series of procedures is followed, which may be taken to represent the respective assay. An assay may include quantitated reagents and established protocols to assess the presence, absence, amount or activity of a biological entity.

In embodiments of the invention involving detection of one or more polypeptides (as opposed to nucleic acid molecules encoding the polypeptides), the detection method may be, for example, an ELISA-based method. As used herein, the term “ELISA” includes an enzyme-linked immunoabsorbent assay that employs an antibody or antigen bound to a solid phase and an enzyme-antigen or enzyme-antibody conjugate to detect and quantify the amount of an antigen (e.g., marker of viral infection of the invention) or antibody present in a sample. A description of the ELISA technique is found in Chapter 22 of the 4th Edition of Basic and Clinical Immunology by D. P. Sites et al., 1982, published by Lange Medical Publications of Los Altos, Calif., and in U.S. Pat. Nos. 3,654,090; 3,850,752; and 4,016,043, the disclosures of which are incorporated herein by reference. ELISA is an assay that can be used to quantify the amount of antigen, proteins, or other molecules of interest in a sample.

The term “binding assay” generally refers to a method of determining the interaction of matter. Hence, some embodiments of a binding assay can be used to qualitatively or quantitatively determine the ability of matter, e.g. a substance, to bind to other matter, e.g. a protein, a nucleic acid or any other substance. Some embodiments of a binding assay can be used to analyse the presence and/or the amount of matter on the basis of binding of the matter to a reagent such as a binding partner that is used in the method/assay. Where a binding assay is based on the use of an immunoglobulin or a proteinaceous binding molecule with\ immunoglobulin-like functions as a binding partner such a method/procedure may also be called an “immunoassay”. In this regard, it is understood that the signals obtained from an immunoassay are a direct result of complexes formed between one or more immunoglobulins or proteinaceous binding molecules with immunoglobulin-like functions and the corresponding composition (i.e., a BK virus polypeptide or antigen) containing the necessary epitope(s) to which the binding partner(s) bind(s). While such an assay may detect the full length composition and the assay result be expressed as a concentration of the composition of interest, the signal from the assay is actually a result of all such “immunoreactive” molecules present the sample. Expression of a polypeptide that is being detected/quantitated may also be determined by means other than an immunoassay, including protein measurements such as dot blots, Western blots, chromatographic methods, mass spectrometry, and nucleic acid measurements such as mRNA quantification.

The term “detect” or “detecting”, as well as the term “determine” or “determining” when used in the context of a serum antibody to BK virus, refers to any method that can be used to detect the presence of a protein or polypeptide associated with a BK virus (e.g., antibodies of a subject that recognize BK virus or an epitope of a polypeptide of BK virus). When used herein in combination with the words “level”, “amount” or “value”, the words “detect”, “detecting”, “determine” or “determining” are understood to generally refer to a quantitative rather than a qualitative level. Accordingly, a method according to the invention includes a quantification of anti-BK virus antibodies—i.e. the amount of serum BK virus antibodies are analyzed. In this regard the words “value,” “amount” and “level” are used interchangeably herein. The exact nature of the “level”, “amount” or “value” depends on the specific design and components of the particular analytical method employed to detect anti-BK virus antibodies. Similarly, the term “detect” or “detecting”, as well as the term “determine” or “determining” when used in the context of a JC virus, refers to any method that can be used to detect the presence of a protein or polypeptide associated with a JC virus (e.g., antibodies of a subject that recognize JC virus or an epitope of a polypeptide of JC virus). When used herein in combination with the words “level”, “amount” or “value”, the words “detect”, “detecting”, “determine” or “determining” are understood to generally refer to a quantitative rather than a qualitative level. Accordingly, a method according to the invention includes a quantification of anti-JC virus antibodies—i.e. the amount of serum JC virus antibodies are analyzed. In this regard the words “value,” “amount” and “level” are used interchangeably herein. The exact nature of the “level”, “amount” or “value” depends on the specific design and components of the particular analytical method employed to detect anti-JC virus antibodies.

A “differential”, “differing” or “altered” level, as used throughout the present application, is observed when a difference in the amount of, e.g., serum antibody to BK virus and/or serum antibody to JC virus of the invention can be analysed by measurement of such. A differential level is for example observed when the amount of a serum antibody to BK virus is lower or higher than that observed from one or more control subjects such that one of skill in the art would consider it to be of statistical significance. As further explained below, in some embodiments the amount of antibody is considered differential or altered when the amount is increased or decreased by about 10% as compared to the control level. In some embodiments, the level is considered differential when it is increased or decreased by about 25%, by about 50%, by about 75%, by about 100%, by about 200%, by about 500% or more, when compared to the control level. In some embodiments a level or an amount is deemed “differential”, “increased” or “decreased” when the amount is increased or decreased by at least about 0.1 fold, as compared to a control level. In some embodiments, the amount is considered differential when it is increased or decreased by at least about 0.2 fold. In some embodiments, the level is considered differential when it is increased or decreased by about a factor of 1, including at least about 2-fold, 3-fold, 4-fold, 5-fold, 10-fold, 100-fold, 1000-fold, 10000-fold, 1000000-fold, etc. as compared to a control level.

In some embodiments, a differential level is measured using a p-value. For instance, when using a p-value, an amount is identified as being different between a first subject or population and second subject or population when the p-value is less than about 0.1, including less than about 0.05, less than about 0.01, less than about 0.005, or even less than about 0.001.

An “effective amount” or a “therapeutically effective amount” of a compound, such as an α₄-integrin blocking agent or a VLA-4 blocking agent, is an amount—either as a single dose or as part of a series of doses—sufficient to provide a therapeutic benefit in the treatment or management of the relevant pathological condition, or to delay or minimize one or more symptoms associated with the presence of the condition. Such a condition may be associated with immunosuppression, e.g. an autoimmune disease or disorder.

The term “occurrence of PML” as used in this disclosure includes a condition having one or more characteristics indicative of the presence of PML. As explained above, the typical characteristic of PML is demyelination in brain tissues. The characteristic of PML generally used in the art for diagnostic purposes is the presence of JC virus DNA in cerebrospinal fluid or a brain biopsy specimen. Further characteristics may be assessed, e.g. visual field testing, ophthalmologic examination and/or cranial magnetic resonance imaging may be performed.

As used in this document, the expression “pharmaceutically acceptable” refers to those active compounds, materials, compositions, carriers, and/or dosage forms which are, within the scope of sound medical judgment, suitable for use in contact with the tissues of human beings and animals without excessive toxicity, irritation, allergic response, or other problems or complications, commensurate with a reasonable benefit risk ratio.

“Plasma” as used in this disclosure refers to acellular fluid found in blood. “Plasma” may be obtained from blood by removing whole cellular material from blood by methods known in the art such as centrifugation or filtration.

The terms “polypeptide” and “protein” refer to a polymer of amino acid residues and are not limited to a certain minimum length of the product. Where both terms are used concurrently, this two-fold naming accounts for the use of both terms side by side in the art.

The term “predicting the risk” as used in the disclosure refers to assessing the probability that a subject will suffer from PML in the future. As will be understood by those skilled in the art, such an assessment is usually not intended to be correct for 100% of the subjects to be investigated. The term, however, requires that a prediction can be made for a statistically significant portion of subjects in a proper and correct manner. Whether a portion is statistically significant can be determined by those skilled in the art using various well known statistic evaluation tools, e.g., determination of confidence intervals, p-value determination. Student's t-test, and Mann-Whitney test. Suitable confidence intervals are generally at least 90%, at least 95%, at least 97%, at least 98%, or at least 99%. Suitable p-values are generally 0.1, 0.05, 0.01, 0.005, or 0.0001. In one embodiment of the disclosed methods, the probability envisaged by the present disclosure allows that the prediction of an increased, normal, or decreased risk will be correct for at least 60%, at least 70%), at least 80%, or at least 90% of the subjects of a given cohort or population. Predictions of risk in a disclosed method relates to predicting whether or not there is an increased risk for PML compared to the average risk for developing PML in a population of subjects rather than giving a precise probability for the risk.

In this regard, the term “prognosis”, commonplace and well-understood in medical and clinical practice, refers to a forecast, a prediction, an advance declaration, or foretelling of the probability of occurrence of a disease state or condition in a subject not (yet) having the respective disease state or condition. In the context of the present invention prognosis refers to the forecast or prediction of the probability as to whether a subject will or will not suffer from PML.

The term “preventing” in the medical physiological context, i.e. in the context of a physiological state, refers to decreasing the probability that an organism contracts or develops an abnormal condition.

Diagnosing, determining, assessing or predicting the “risk of occurrence” of PML is understood to refer to an analysis of a relative degree of a risk when compared to a healthy individual. The term “risk of occurrence” refers to the likelihood or probability that PML will occur in a subject. Without being bound by theory, PML is thought to be a reactivation of latent infection with JC virus (JCV). While a general susceptibility to occurrence of PML is linked to the presence of JCV in a subject's organism, the mere presence of JCV in an organism does not indicate whether there is or will be an infection of oligodendrocytes with JCV, which leads to demyelination.

Hence, there can generally only be an elevated risk of PML occurrence if JCV is present in an organism. However, for purpose of the present invention the actual risk level is also determined on the basis of the level of serum antibody to BK virus in a subject. In the context of diagnosis and risk assessment, determining/predicting the risk of occurrence of PML is a relative assessment whether a particular subject is at a higher risk or not at a higher risk of suffering from PML at a point of time in the future, when compared to a healthy subject or to an average subject that is in an otherwise comparable physiological condition.

The term “reducing the risk”, as used in this document, means to lower the likelihood or probability of a disease state or condition, e.g., PML, from occurring in a subject, especially when the subject is predisposed to such or at risk of contracting a disease state or condition, e.g., PML.

The terms “screening subjects”, “screening individuals” or “screening patients” in the context of risk assessment refers to a method or process of determining if a subject/patient or a plurality of subjects/patients is or is not likely to suffer from a disease or disorder such as PML, or has or does not have an increased risk of developing a disease or disorder. “Screening compounds” and a “screening assay” means a process or method used to characterize or select compounds based upon their activity from a collection of compounds.

“Serum” as used in this disclosure, refers to components of blood that do not define a cell, such as a leukocyte, and that do not define a clotting factor. Serum includes the fraction of plasma obtained after plasma or blood is permitted to clot and the clotted fraction is removed.

The terms “stratifying” and “stratification” as used herein indicate in one aspect that individuals are assigned to groups with similar characteristics such as at a similar risk level of developing PML. As an illustrative example, individuals may be stratified into risk categories. The terms “stratifying” and “stratification” as used herein indicate in another aspect that an individual is assigned to a certain group according to characteristics matching the respective group such as a corresponding risk level of developing PML. The groups may be, for example, for testing, prescribing, suspending or abandoning any one or more of a drug, surgery, diet, exercise, or intervention. Accordingly, in some embodiments of a method or use according to the invention a subject may be stratified into a subgroup of a clinical trial of a therapy. As explained in the following, in the context of the present invention, serum antibody to BK virus, and/or serum antibody to JC virus may be used for PML risk stratification.

The terms “stratifying” and “stratification” according to the invention generally include identifying subjects that require an alteration of their current or future therapy. The term includes assessing, e.g., determining, which therapy a subject likely to suffer from PML is in need of. Hence, in the context of the present invention, stratification may be based on the probability (or risk) of developing PML. A method or use according to the invention may also serve in stratifying the probability of the risk of PML or the risk of any PML-related condition for a subject. A method of stratifying a subject for PML therapy according to the invention includes detecting the presence and/or amount of serum antibody to BK virus in the subject. As explained above, in some embodiments, presence of serum antibody to BK virus can be used to screen risk patients which are at a reduced risk or have a reduced predisposition to develop PML.

The term “subject” as used herein, also addressed as an individual, refers to a human or non-human animal, generally a mammal. A subject may be a mammalian species such as a rabbit, a mouse, a rat, a Guinea pig, a hamster, a dog, a cat, a pig, a cow, a goat, a sheep, a horse, a monkey, an ape or a human. Thus, the methods, uses and compositions described in this document are applicable to both human and veterinary disease. As explained in more detail below, the sample has been obtained from the subject. It is thus understood that conclusions drawn from expression levels in the sample and decisions based thereon concern the subject from whom/which the subject has been taken. Further, while a subject is typically a living organism, the invention described in this document may also be used in post-mortem analysis. Where the subject is a living human who is receiving medical care for a disease or condition, it is also addressed as a “patient”.

The term “susceptibility” as used in this document refers to the proneness of a subject towards the development of a certain state or a certain condition such as a pathological condition, including a disease or disorder, in particular PML, or towards being less able to resist a particular state than the average individual. Susceptibility to PML is in particular dependent on the presence of JCV in an organism, and is further influenced, as newly described herein, based upon the absence and/or decreased level relative to a threshold of serum antibody to BK virus in an organism.

As used herein, the term “antibody” refers to a protein that includes at least one immunoglobulin variable region, e.g., an amino acid sequence that provides an immunoglobulin variable domain or an immunoglobulin variable domain sequence. For example, an antibody can include a heavy (H) chain variable region (abbreviated herein as VH), and a light (L) chain variable region (abbreviated herein as VL). In another example, an antibody includes two heavy (H) chain variable regions and two light (L) chain variable regions. The term “antibody” encompasses antigen-binding fragments of antibodies (e.g., single chain antibodies, Fab fragments, F(ab′)2 fragments, Fd fragments, Fv fragments, and dAb fragments) as well as complete antibodies, e.g., intact and/or full length immunoglobulins of types IgA, IgG (e.g., IgG1, IgG2, IgG3, IgG4), IgE, IgD, IgM (as well as subtypes thereof). The light chains of the immunoglobulin may be of types kappa or lambda. In one embodiment, the antibody is glycosylated. An antibody can be functional for antibody-dependent cytotoxicity and/or complement-mediated cytotoxicity, or may be non-functional for one or both of these activities.

The VH and VL regions can be further subdivided into regions of hypervariability, termed “complementarity determining regions” (“CDR”), interspersed with regions that are more conserved, termed “framework regions” (FR). The extent of the FR's and CDR's has been precisely defined (see, Kabat, E. A. et al. (1991) Sequences of Proteins of Immunological Interest, Fifth Edition, U.S. Department of Health and Human Services, NIH Publication No. 91-3242; and Chothia. C. et al (1987) J. Mol. Biol. 196:901-917). Kabat definitions are used herein. Each VH and VL is typically composed of three CDR's and four FR's, arranged from amino-terminus to carboxyl-terminus in the following order: FR1, CDR1, FR2, CDR2, FR3, CDR3, FR4.

An “immunoglobulin domain” refers to a domain from the variable or constant domain of immunoglobulin molecules Immunoglobulin domains typically contain two β-sheets formed of about seven β-strands, and a conserved disulphide bond (see, e.g., A. F. Williams and A. N. Barclay (1988) Ann Rev. Immunol. 6:381-405). An “immunoglobulin variable domain sequence” refers to an amino acid sequence that can form a structure sufficient to position CDR sequences in a conformation suitable for antigen binding. For example, the sequence may include all or part of the amino acid sequence of a naturally-occurring variable domain. For example, the sequence may omit one, two, or more N- or C-terminal amino acids, internal amino acids, may include one or more insertions or additional terminal amino acids, or may include other alterations. In one embodiment, a polypeptide that includes an immunoglobulin variable domain sequence can associate with another immunoglobulin variable domain sequence to form a target binding structure (or “antigen binding site”), e.g., a structure that interacts with TWEAK or a TWEAK receptor.

The VH or VL chain of the antibody can further include all or part of a heavy or light chain constant region, to thereby form a heavy immunoglobulin chain (HC) or light immunoglobulin chain (LC), respectively. In one embodiment, the antibody is a tetramer of two heavy immunoglobulin chains and two light immunoglobulin chains. The heavy and light immunoglobulin chains can be connected by disulfide bonds. The heavy chain constant region typically includes three constant domains, CH1, CH2, and CH3. The light chain constant region typically includes a CL domain. The variable region of the heavy and light chains contains a binding domain that interacts with an antigen. The constant regions of the antibodies typically mediate the binding of the antibody to host tissues or factors, including various cells of the immune system (e.g., effector cells) and the first component (Clq) of the classical complement system.

One or more regions of an antibody can be human, effectively human, or humanized. For example, one or more of the variable regions can be human or effectively human. For example, one or more of the CDRs, e.g., HC CDR1, EC CDR2, HC CDR3, LC CDR1, LC CDR2, and LC CDR3, can be human. Each of the light chain CDRs can be human. HC CDR3 can be human. One or more of the framework regions can be human, e.g., FR1, FR2, FR3, and FR4 of the HC or LC. In one embodiment, all the framework regions are human, e.g., derived from a human somatic cell, e.g., a hematopoietic cell that produces immunoglobulins, or a non-hematopoietic cell. In one embodiment, the human sequences are germline sequences, e.g., encoded by a germline nucleic acid. One or more of the constant regions can be human, effectively human, or humanized. In another embodiment, at least 70, 75, 80, 85, 90, 92, 95, or 98% of the framework regions (e.g., FR1, FR2, and FR3, collectively, or FR1, FR2, FR3, and FR4, collectively) or the entire antibody can be human, effectively human, or humanized. For example, FR1, FR2, and FR3 collectively can be at least 70, 75, 80, 85, 90, 92, 95, 98, or 99% identical, or completely identical, to a human sequence encoded by a human germline segment.

An “effectively human” immunoglobulin variable region is an immunoglobulin variable region that includes a sufficient number of human framework amino acid positions such that the immunoglobulin variable region does not elicit an immunogenic response in a normal human. An “effectively human” antibody is an antibody that includes a sufficient number of human amino acid positions such that the antibody does not elicit an immunogenic response in a normal human.

A “humanized” immunoglobulin variable region is an immunoglobulin variable region that is modified such that the modified form elicits less of an immune response in a human than does the non-modified form, e.g., is modified to include a sufficient number of human framework amino acid positions such that the immunoglobulin variable region does not elicit an immunogenic response in a normal human. Descriptions of “humanized” immunoglobulins include, for example, U.S. Pat. Nos. 6,407,213 and 5,693,762. In some cases, humanized immunoglobulins can include a non-human amino acid at one or more framework amino acid positions.

The terms “treatment” and “treating” as used herein, refer to a prophylactic or preventative measure having a therapeutic effect and preventing, slowing down (lessen), or at least partially alleviating or abrogating an abnormal, including pathologic, condition in the organism of a subject. Those in need of treatment include those already with the disorder as well as those prone to having the disorder or those in whom the disorder is to be prevented (prophylaxis). Generally a treatment reduces, stabilizes, or inhibits progression of a symptom that is associated with the presence and/or progression of a disease or pathological condition. The term “administering” relates to a method of incorporating a compound into cells or tissues of a subject. The term “therapeutic effect” refers to the inhibition or activation of factors causing or contributing to the abnormal condition. A therapeutic effect relieves to some extent one or more of the symptoms of an abnormal condition or disease. The term “abnormal condition” refers to a function in the cells or tissues of an organism that deviates from their normal functions in that organism. An abnormal condition can inter alia relate to cell proliferation, cell differentiation, or cell survival.

The term “VLA-4 blocking agent” refers to a molecule that binds to the VLA-4 antigen on the surface of a leukocyte with sufficient specificity to inhibit the VLA-4/VCAM-1 interaction. In some embodiments the blocking agent binds to VLA-4 integrin with a K_{D} of less than 10⁻⁶ M. A VLA-4 blocking agent may be a VLA-4 binding antibody such as an anti-VLA-4 immunoglobulin or a fragment of an anti-VLA-4 immunoglobulin. A VLA-4 blocking agent generally inhibits the migration of leukocytes from the blood to the central nervous system by disrupting adhesion between the T-cell and endothelial cells. This is believed to result in the reduction of proinflammatory cytokines, and thus the reduction of the occurrence of pathologic inflammatory disease within the CNS. Examples of a VLA-4 blocking agent include, but are not limited to, Natalizumab (Biogcn, U.S. Pat. No. 5,840,299), monoclonal immunoglobulins HP2/1, HP1/3 (Elices et al. Cell (1990) 60, 577-584), HP 1/2 (Sanchez-Madrid et al, Eur J Immunol (1986) 16, 1 343-1 349), humanized HP 1/2 (U.S. Pat. No. 6,602,503), HP1/7, HP2/4, B-5G10, TS2/16 (Pulido et al, J Biol. Chem. (1991) 266, 16, 10241-5), monoclonal immunoglobulin L25 (Becton Dickinson GmBH, Germany), P4C2 (Abeam, Cambridge, U K), and AJM300 (Ajinomoto, Japan), and recombinant anti-VLA4 immunoglobulins as described in U.S. Pat. No. 6,602,503 and U.S. Pat. No. 7,829,092. In one embodiment, the VLA-blocking agent is specific for CD49d (α₄-integrin). As a further example, a VLA-4 blocking agent may also be a VLA-4 antagonist that differs from an antibody such as an immunoglobulin, illustrative examples of such an antagonist are the low molecular weight compound SB-683699 (GlaxoSmithKline, Middlesex, UK), which is a dual α₄ antagonist, a CS-1 peptidomimetic (U.S. Pat. Nos. 5,821,231; 5,869,448; 5,869,448; 5,936,065; 6,265.572; 6,288,267; 6,365,619; 6.423,728; 6,426,348; 6,458,844; 6,479,666; 6,482,849; 6,596,752; 6,667,331; 6,668,527; 6,685,617; 6,903,128; and 7,015,216), a phenylalanine derivative (U.S. Pat. Nos. 6,197,794; 6,229,011; 6,329,372; 6,388,084; 6,348,463; 6,362,204; 6,380,387; 6,445,550; 6,806,365; 6,835,738; 6,855,706; 6,872,719; 6,878,718; 6,911,451; 6,916,933; 7,105,520; 7,153,963; 7,160,874; 7,193,108; 7,250,516; and 7,291,645) alphafeto protein (U.S. Pat. Pub. No. 2010/0150915), a beta-amino acid compound (U.S. Pat. Pub. Nos. 2004/0229859 and 2006/021 1630), a semi-peptide compound (U.S. Pat. No. 6,376,538), Leu-Asp-Val tripeptide (U.S. Pat. No. 6,552,216), or a pegylated molecule as described in U.S. patent application 2007/066533 and U.S. Pat. No. 6,235,711.

An “α₄-integrin blocking agent” refers to a molecule that binds to the α₄-subunit of integrins with a specificity and an affinity and/or k_{off} rate that is sufficient to inhibit the interaction with a physiological ligand such as MAdCAM-1, VCAM-1 or CS-1 of the respective integrin. In some embodiments the blocking agent binds to an integrin that has an α₄-subunit with an affinity constant of at least about 10⁻⁵ M. In some embodiments the affinity constant has a value of at least about 10⁻⁶ M. The binding affinity may in some embodiments be of a KD of about 0.1 nM or below, in some embodiments the KD may be below 10 picomolar (pM). An α₄-integrin blocking agent may in some embodiments bind to VLA-4 integrin. In some embodiments the α₄-integrin blocking agent binds to LPAM-1 integrin. In some embodiments the α₄-integrin blocking agent binds to both VLA-4 and LPAM-1 integrins. An α₄-integrin blocking agent may be an α₄-integrin binding antibody such as an anti-α₄-integrin immunoglobulin or a fragment of an anti-α₄-integrin immunoglobulin. Examples of an α₄-integrin blocking agent include, but are not limited to, monoclonal immunoglobulins Natalizumab (Biogen, U.S. Pat. No. 5,840,299), Vedolizumab (Millennium Pharmaceuticals, Cambridge, U.S.), HP2/1, HP1/3 (Elices et al, Cell (1990) 60, 577-584), HP1/2 (Sanchez-Madrid et al. Eur J Immunol (1986) 16, 1343-1349), humanized HP1/2 (U.S. Pat. No. 6,602,503), HP1/7, HP2/4, B-5G10, Max68P (Becton Dickinson GmBH, Germany), L25 (Becton Dickinson GmBH, Germany), P₄C₂ (Abeam, Cambridge, UK), R1-2 (BD Biosciences) and AJM300 (Ajinomoto, Japan).

The terms “synergy,” “synergistic,” or “synergistic effect” as used herein describe an effect which has a magnitude that is greater than additive.

As used herein, the terms “prevent,” “preventing” and “prevention” in the context of the administration of a therapy to a subject refer to the prevention or inhibition of the recurrence, onset, and/or development of a condition (e.g., PML) in a subject resulting from the administration of a therapy (e.g., a prophylactic agent), or a combination of therapies (e.g., a combination of prophylactic agents).

By “proliferative disease” or “cancer” as used herein is meant, a disease, condition, trait, genotype or phenotype characterized by unregulated cell growth or replication as is known in the art; including leukemias, for example, acute myelogenous leukemia (AML), chronic myelogenous leukemia (CML), acute lymphocytic leukemia (ALL), and chronic lymphocytic leukemia, AIDS related cancers such as Kaposi's sarcoma; breast cancers; bone cancers such as Osteosarcoma, Chondrosarcomas, Ewing's sarcoma, Fibrosarcomas, Giant cell tumors, Adamantinomas, and Chordomas; Brain cancers such as Meningiomas, Glioblastomas, Lower-Grade Astrocytomas, Oligodendrocytomas, Pituitary Tumors, Schwannomas, and Metastatic brain cancers; cancers of the head and neck including various lymphomas such as mantle cell lymphoma, non-Hodgkins lymphoma, adenoma, squamous cell carcinoma, laryngeal carcinoma, gallbladder and bile duct cancers, cancers of the retina such as retinoblastoma, cancers of the esophagus, gastric cancers, multiple myeloma, ovarian cancer, uterine cancer, thyroid cancer, testicular cancer, endometrial cancer, melanoma, colorectal cancer, lung cancer, bladder cancer, prostate cancer, lung cancer (including non-small cell lung carcinoma), pancreatic cancer, sarcomas, Wilms' tumor, cervical cancer, head and neck cancer, skin cancers, nasopharyngeal carcinoma, liposarcoma, epithelial carcinoma, renal cell carcinoma, gallbladder adeno carcinoma, parotid adenocarcinoma, endometrial sarcoma, multidrug resistant cancers; and proliferative diseases and conditions, such as neovascularization associated with tumor angiogenesis, macular degeneration (e.g., wet/dry AMD), corneal neovascularization, diabetic retinopathy, neovascular glaucoma, myopic degeneration and other proliferative diseases and conditions such as restenosis and polycystic kidney disease, and other cancer or proliferative disease, condition, trait, genotype or phenotype that can respond to the modulation of disease related gene expression in a cell or tissue, alone or in combination with other therapies.

As used herein, “neoplasia” means a disease state of a human or an animal in which there are cells and/or tissues which proliferate abnormally. Neoplastic conditions include, but are not limited to, cancers, sarcomas, tumors, leukemias, lymphomas, and the like. A neoplastic condition refers to the disease state associated with the neoplasia. Lung cancer, colon cancer and ovarian cancer are examples (non-limiting) of a neoplastic condition.

A “cancer” in a subject refers to the presence of cells possessing characteristics typical of cancer-causing cells, such as uncontrolled proliferation, immortality, metastatic potential, rapid growth and proliferation rate, and certain characteristic morphological features. Often, cancer cells will be in the form of a tumor, but such cells may exist alone within a subject, or may be a non-tumorigenic cancer cell, such as a leukemia cell. Examples of cancer include but are not limited to breast cancer, a melanoma, adrenal gland cancer, biliary tract cancer, bladder cancer, brain or central nervous system cancer, bronchus cancer, blastoma, carcinoma, a chondrosarcoma, cancer of the oral cavity or pharynx, cervical cancer, colon cancer, colorectal cancer, esophageal cancer, gastrointestinal cancer, glioblastoma, hepatic carcinoma, hepatoma, kidney cancer, leukemia, liver cancer, lung cancer, lymphoma, non-small cell lung cancer, osteosarcoma, ovarian cancer, pancreas cancer, peripheral nervous system cancer, prostate cancer, sarcoma, salivary gland cancer, small bowel or appendix cancer, small-cell lung cancer, squamous cell cancer, stomach cancer, testis cancer, thyroid cancer, urinary bladder cancer, uterine or endometrial cancer, and vulval cancer.

As used herein, the term “tumor” means a mass of transformed cells that are characterized by neoplastic uncontrolled cell multiplication and at least in part, by containing angiogenic vasculature. The abnormal neoplastic cell growth is rapid and continues even after the stimuli that initiated the new growth has ceased. The term “tumor” is used broadly to include the tumor parenchymal cells as well as the supporting stroma, including the angiogenic blood vessels that infiltrate the tumor parenchymal cell mass. Although a tumor generally is a malignant tumor, i.e., a cancer having the ability to metastasize (i.e. a metastatic tumor), a tumor also can be nonmalignant (i.e. non-metastatic tumor). Tumors are hallmarks of cancer, a neoplastic disease the natural course of which is fatal. Cancer cells exhibit the properties of invasion and metastasis and are highly anaplastic.

The phrase “pharmaceutically acceptable carrier” refers to a carrier for the administration of a therapeutic agent. Exemplary carriers include saline, buffered saline, dextrose, water, glycerol, ethanol, and combinations thereof. For drugs administered orally, pharmaceutically acceptable carriers include, but are not limited to pharmaceutically acceptable excipients such as inert diluents, disintegrating agents, binding agents, lubricating agents, sweetening agents, flavoring agents, coloring agents and preservatives. Suitable inert diluents include sodium and calcium carbonate, sodium and calcium phosphate, and lactose, while corn starch and alginic acid are suitable disintegrating agents. Binding agents may include starch and gelatin, while the lubricating agent, if present, will generally be magnesium stearate, stearic acid or talc. If desired, the tablets may be coated with a material such as glyceryl monostearate or glyceryl distearate, to delay absorption in the gastrointestinal tract.

Various methodologies of the instant invention include a step that involves comparing a value, level, feature, characteristic, property, etc. to a “suitable control”, referred to interchangeably herein as an “appropriate control”. A “suitable control” or “appropriate control” is a control or standard familiar to one of ordinary skill in the art useful for comparison purposes. In one embodiment, a “suitable control” or “appropriate control” is a value, level, feature, characteristic, property, etc. determined prior to performing a treatment and/or agent administration methodology, as described herein. For example, a transcription rate, mRNA level, translation rate, protein level, biological activity, cellular characteristic or property, genotype, phenotype, etc. can be determined prior to introducing a treatment and/or agent of the invention into a cell or organism. In another embodiment, a “suitable control” or “appropriate control” is a value, level, feature, characteristic, property, etc. determined in a cell or organism, e.g., a control or normal cell or organism, exhibiting, for example, normal traits. In yet another embodiment, a “suitable control” or “appropriate control” is a predefined value, level, feature, characteristic, property, etc.

As used herein, the term “compound” refers to small molecules. Examples of such small molecules would include low molecular weight molecules. Other examples of compounds include molecules that are generated by organic synthesis, and low molecular weight molecules that are metabolites or anti-metabolites. In one embodiment, compounds can be administered directly to patients, or can be conjugated to antibodies or protein-based agents. In another embodiment, compounds can be administered in combination with other agents. In a specific embodiment, the agent administered in combination with a compound is an antibody or antibody-based therapeutic.

As used herein, the term “in combination” in the context of the administration of a therapy to a subject refers to the use of more than one therapy for therapeutic benefit. The term “in combination” in the context of the administration can also refer to the prophylactic use of a therapy to a subject when used with at least one additional therapy. The use of the term “in combination” does not restrict the order in which the therapies (e.g., a first and second therapy) are administered to a subject. A therapy can be administered prior to (e.g., 1 minute, 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks before), concomitantly with, or subsequent to (e.g., 1 minute, 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks after) the administration of a second therapy to a subject which had, has, or is susceptible to cancer. The therapies are administered to a subject in a sequence and within a time interval such that the therapies can act together. In a particular embodiment, the therapies are administered to a subject in a sequence and within a time interval such that they provide an increased benefit than if they were administered otherwise. Any additional therapy can be administered in any order with the other additional therapy.

Ranges provided herein are understood to be shorthand for all of the values within the range. For example, a range of 1 to 50 is understood to include any number, combination of numbers, or sub-range from the group consisting 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50. Concentrations, amounts, cell counts, percentages and other numerical values may be presented herein in a range format. It is to be understood that such range format is used merely for convenience and brevity and should be interpreted flexibly to include not only the numerical values explicitly recited as the limits of the range but also to include all the individual numerical values or sub-ranges encompassed within that range as if each numerical value and sub-range is explicitly recited.

Unless specifically stated or obvious from context, as used herein, the term “or” is understood to be inclusive. Unless specifically stated or obvious from context, as used herein, the terms “a”, “an”, and “the” are understood to be singular or plural.

Unless specifically stated or obvious from context, as used herein, the term “about” is understood as within a range of normal tolerance in the art, for example within 2 standard deviations of the mean. About can be understood as within 10%, 9%, 8%, 7%, 6%, 5%, 4%, 3%, 2%, 1%, 0.5%, 0.1%, 0.05%, or 0.01% of the stated value. Unless otherwise clear from context, all numerical values provided herein are modified by the term about.

The terms “comprising”, “including,” containing”, “having” etc. shall be read expansively or open-ended and without limitation. Singular forms such as “a”, “an” or “the” include plural references unless the context clearly indicates otherwise. Thus, for example, reference to a “vector” includes a single vector as well as a plurality of vectors, either the same—e.g. the same operon—or different. Likewise reference to “cell” includes a single cell as well as a plurality of cells. Unless otherwise indicated, the term “at least” preceding a series of elements is to be understood to refer to every element in the series. The terms “at least one” and “at least one of” include for example, one, two, three, four, or five or more elements. It is furthermore understood that slight variations above and below a stated range can be used to achieve substantially the same results as a value within the range. Also, unless indicated otherwise, the disclosure of ranges is intended as a continuous range including every value between the minimum and maximum values.

The scope and meaning of any use of a term will be apparent from the specific context in which the term is used. Certain further definitions for selected terms used throughout this document are given in the appropriate context of the detailed description, as applicable. Unless otherwise defined, all other scientific and technical terms used in the description, figures and claims have their ordinary meaning as commonly understood by one of ordinary skill in the art.

### BRIEF DESCRIPTION OF THE DRAWINGS

FIG. 1 shows that an inverse correlation was detected between BK and JC virus serum capsid antibody levels. To generate the plotted data, 200 serum samples were tested in a BKV and JCV VLP-based ELISA, and the scatter plot was constructed of BK vs JC optical density values. In the Pearson's correlation coefficient test, the optical density values were shown to be significantly negatively correlated (R=−0.273), such that samples strongly reactive in the BK VLP ELISA tended to be weakly reactive in the JC VLP ELISA, and vice versa.

FIG. 2 shows that competitive inhibition of BK and JC capsid antibody responses was observed by homologous but not heterologous VP1 protein, demonstrating the lack of serological cross-reactivity. In particular, a scatter plot of percent competitive inhibition of BKV and JCV reactivity of 23 human sera by BKV and JCV VLP protein is shown. Serum samples were tested in the BKV (circle) or JCV (triangle) VLP ELISA in the presence of 4 μg/ml of BKV or 4 μg/ml of JCV VLP protein. Percent inhibition by each VLP was calculated as 1−OD_{competing VLP}/OD_{buffer control}×100. The percent inhibition by BKV VLP protein (y-axis) is plotted versus percent inhibition by JCV VLP protein (x-axis). The horizontal and vertical lines mark 50% inhibition by BKV and JCV VLP protein, respectively. Reactivity in the BKV VLP ELISA (circle) was blocked by >50% by incubation with BK VP1 protein in 21 of 23 serum samples, whereas JCV VLP protein blocked reactivity by <10% in 22 of the 23 samples. Conversely, reactivity in the JCV ELISA (triangle) was blocked by >50% by incubation with JCV VP1 protein in 22 of 23 serum samples, whereas BK VP1 protein blocked <5% of the reactivity in 23 of 23 samples.

FIG. 3 depicts the age-specific seroprevalence of Merkel cell polyomavirus, BK virus and JC virus. To generate the chart, serum samples from 945 individuals recruited from hospital-based general and subspecialty outpatient clinics were tested for capsid antibodies to Merkel cell, BK and JC polyomaviruses in virus-like particle based ELISA assays. The distribution of reactivity of serum samples from children less than 10 years of age was used to set cut points for seropositivity, and results are displayed as the percent positive in 10-year age groups. Among children less than 10 years of age, the seroprevalence to JC virus was approximately 10%, while that to BK virus was greater than 60%. These data illustrate the earlier exposure to BK virus compared to JC virus observed in the general population, supporting the presence of immunity to BK virus at the time most subjects are first infected with JC virus. Without wishing to be bound by theory, the inverse relationship observed between BK and JC antibody levels appears to be a consequence of the sequential nature of infection with the two viruses.

FIG. 4 shows the distribution of OD values in BKV1, BKV4 and JCV ELISA by case control status during 6 month windows between 0.5 and 2 years before diagnosis of PML.

FIGS. 5A to 5C show decision tree analysis for classification of cases and controls. FIG. 5A shows the output of decision tree analysis for classification of cases and controls using average data values at 0.5-2 years before PML diagnosis for 4 parameters (BKV 1, BKV 4 and JCV ELISA, and CD4+ T cell count). FIG. 5B shows the decision tree of FIG. 5A. FIG. 5C shows a decision tree analysis of FIG. 5A with the number of nodes reduced from 11 to 7.

FIG. 6 shows a boxplot of % BKV4 inhibition by case-control status at 1-1.5 years before PML.

### DETAILED DESCRIPTION OF THE INVENTION

The present invention relates, at least in part, to the unexpected observation that BK virus serology can be used for risk stratification of progressive multifocal leukoencephalopathy (PML) in patients, for example those at risk due to human immunodeficiency virus (HIV)-associated immunosuppression or due to treatment with natalizumab (Tysabri) and other humanized monoclonal antibodies targeting immune functions.

In particular, the invention provides a method for stratifying the risk of PML in patients with HIV and/or receiving natalizumab or other humanized monoclonal antibody therapies that target the immune system. The invention is grounded in empirical observations of serological responses to these viruses in human populations, namely, the observation that there is an inverse relationship between levels of antibody to these two closely related human polyomaviruses. The invention is further based on the notion that cellular immune responses to the viruses confer partial cross-protection. In support of this notion. there is limited experimental work identifying cross reactive T cell epitopes in the two viruses. Newly identified and appreciated herein is that a low level of JC antibody in a person exposed to the virus is likely to be due to partial protection conferred by cross-reactive BK virus-specific T cell responses, and the extent of this protection is proportional to the BK virus antibody titer. Also important for purpose of this invention is the converse situation where a high level of JC antibody in a person exposed to the virus is explained by the absence of significant cross-protection by BK virus T cells, as reflected in a low serum antibody level to BK virus. Individuals with a low level of antibody to BK virus and consequently little cross-protection against JC virus will have a more robust JC virus infection and carry a lifelong greater risk of JC virus-associated PML when infected with HIV or treated with natalizumab or other humanized monoclonal antibodies targeting immune functions. Seroepidemiology studies have established the fact that most individuals are exposed to BK virus before they encounter JC virus, and thus these individuals are expected to have cross-protective immunity at the time they are exposed to JC virus. As a result, they mount modest but not high levels of JC virus antibody and have a low body burden of JC virus infection, putting them at low risk of PML. The risk of PML and the incidence of PML in patients receiving monoclonal antibody therapy is low in the general population because most individuals are partially protected by prior BK virus infection when they become infected with JC virus. Since BK virus serum antibody levels are correlated with cross protective T cell responses, detection of BK antibody can be used to identify those individuals at risk of PML. By combining measurement of JC virus antibody level (seropositive) and BK virus antibody level (low level), the risk for PML can be more precisely defined than currently possible with the use of JC virus serology alone.

The level of BK virus antibody that will identify JC virus seropositive patients with the greatest risk of PML is determined empirically by testing serum samples from, for example, natalizumab-treated patients who develop PML and comparing with that observed in treated patients who remain disease-free. Standard statistical analyses of the data allow definition of a clinically relevant level. In certain aspects, the invention provides a serological test that can be performed on all patients (or a subset thereof) eligible to be treated with natalizumab or other humanized monoclonal antibodies targeting immune functions, to determine the risk of such subject(s) developing PML, thereby allowing for an informed decision about the risks and benefits of treatment to be made. The serological testing can also be performed during the course of treatment to determine if risk changes over time. The relevance of such assessment as described herein is likely to expand as this category of drugs is further developed and more widely used. The serological testing can be performed on HIV infected patients to determine their level of risk for PML and the need for close monitoring for early signs of disease to implement therapeutic interventions.

Because PML is caused by JC virus, exposure to JC virus is a necessary risk factor. In fact, JC virus seropositive individuals have been shown to have a much higher risk of PML than JC seronegative patients (Gorelik et al., 2010; Sorensen et al., 2012). However, the seroprevalence to JC virus ranges from 50-70% in the general population and the occurrence of PML is rare. Additional factors that identify at risk patients are the duration of monoclonal antibody therapy and prior use of immunosuppressive therapy. More recently, it has been reported that the titer of antibody to JC virus allows more precise risk stratification. Nevertheless, between 30-50% of individuals are at some risk of PML when treated with natalizumab or other monoclonal antibody therapies.

A serious adverse event associated with natalizumab, and less commonly other drugs in this category, is progressive multifocal leukoencephalopathy (PML), a severe neurological disease caused by JC virus. Although natalizumab is the best drug currently on the market to treat multiple sclerosis (natalizumab has also been approved for use in Crohn's disease and clinical testing of natalizumab for use against neoplastic conditions is in progress), the use of natalizumab has been significantly limited by the risk of PML in subjects administered the drug. Research to identify individuals who are at risk of PML is a high priority. This invention concerns the use of BK virus serology in conjunction with JC virus serology, to further refine risk stratification for natalizumab-associated PML. The invention specifies that the risk of PML will be greatest in JC virus seropositive patients with a low titer of serum antibody to BK virus capsids. The particular level of antibody which will identify individuals at risk of PML needs to be established empirically by comparing levels in natalizumab-treated patients who develop PML with that in treated patients who remain disease free.

### BK Virus

The BK virus is a member of the polyomavirus family. Past infection with the BK virus is widespread, but significant consequences of infection are uncommon, with the exception of the immunocompromised and the immunosuppressed.

The BK virus was first isolated in 1971 from the urine of a renal transplant patient, initials B.K. (Gardner S D, Field A M, Coleman D V, Hulme B (June 1971). “New human papovavirus (B.K.) isolated from urine after renal transplantation”. Lancet 1 (7712): 1253-7) The BK virus is similar to another virus called the JCV since their genomes share 75% sequence similarity. Both of these viruses can be identified and differentiated from each other by carrying out serological tests using specific antibodies or by using a PCR based genotyping approach. Surveys of human population infected with BK virus have identified 4 major viral genotypes, I, II, III and IV (Jin, L., P. E. Gibson, J. C. Booth, and J. P. Clewley. 1993. Genomic typing of BK virus in clinical specimens by direct sequencing of polymerase chain reaction products. J. Med. Virol. 41:11-17.), which correspond to unique serotypes (Knowles, W. A., P. E. Gibson, and S. D. Gardner. 1989. Serological typing scheme for BK-like isolates of human polyomavirus. J. Med. Virol. 28:118-123 and Pastrana D V, Ray U, Magaldi T G, Schowalter R M, çuburu N, Buck C B. BK polyomavirus genotypes represent distinct serotypes with distinct entry tropism. J Virol. 2013 September; 87(18):10105-13)

The GenBank reference sequence for the BK virus type I genome is NCBI Reference Sequence: NC_001538.1.

The BK virus rarely causes disease since many people who are infected with this virus are asymptomatic. If symptoms do appear, they tend to be mild: respiratory infection or fever. These are known as primary BK infections.

The virus then disseminates to the kidneys and urinary tract where it persists for the life of the individual. It is thought that up to 80% of the population contains a latent form of this virus, which remains latent until the body undergoes some form of immunosuppression. Typically, this is in the setting of kidney transplantation or multi-organ transplantation (Gupta G, Shapiro R, Thai N, Randhawa P S, Vats A (August 2006). “Low incidence of BK virus nephropathy after simultaneous kidney pancreas transplantation”. Transplantation 82 (3): 382-8). Presentation in these immunocompromised individuals is much more severe. Clinical manifestations include renal dysfunction (seen by a progressive rise in serum creatinine), and an abnormal urinalysis revealing renal tubular cells and inflammatory cells.

It is not known how this virus is transmitted. It is known, however, that the virus is spread from person to person, and not from an animal source. It has been suggested that this virus may be transmitted through respiratory fluids or urine, since infected individuals periodically excrete virus in the urine. A survey of 400 healthy blood donors was reported as showing that 82% were positive for BK virus (Egli A, Infanti L, Dumoulin A, et al. (2009). “Prevalence of polyomavirus BK and JC infection and replication in 400 healthy blood donors”. J Infect Dis 199 (6): 837-46).

In some renal transplant patients, the necessary use of immunosuppressive drugs has the side-effect of allowing the virus to replicate within the graft, a disease called BK nephropathy (Fishman, J. A. (2002). “BK Virus Nephropathy—Polyomavirus Adding Insult to Injury”. New England Journal of Medicine 347 (7): 527-530). From 1-10% of renal transplant patients progress to BK virus nephropathy (BKVN) and up to 80% of these patients lose their grafts. The onset of nephritis can occur as early as several days post-transplant to as late as 5 years.

It is also associated with ureteral stenosis and interstitial nephritis. In bone marrow transplant recipients it is notable as a cause for hemorrhagic cystitis.

The virus can be diagnosed by a BKV blood test or a urine test for decoy cells, in addition to carrying out a biopsy, e.g., in the kidneys. PCR techniques are often carried out to identify the virus (Bista, B R; Ishwad, C; Wadowsky, R M; Manna, P; Randhawa, P S; Gupta, G; Adhikari, M; Tyagi, R et al. (2007). “Development of a Loop-Mediated Isothermal Amplification Assay for Rapid Detection of BK Virus”. Journal of clinical microbiology 45 (5): 1581-7).

The cornerstone of therapy is reduction in immunosuppression. A recent surge in BKVN correlates with use of potent immunosuppressant drugs, such as tacrolimus and mycophenolate mofetil (MMF). Studies have not shown any correlation between BKVN and a single immunosuppressive agent but rather the overall immunosuppressive load.

No guidelines or drug levels and doses exist for proper reduction of immunosuppressants in BKVN Most common methods include: 1. Withdrawal of MMF or tacrolimus; 2. Replacement of tacrolimus by cyclosporine; 3. Overall reduction of immunosuppressive load; 4. Some cyclosporine trough levels reported to be reduced to 100-150 ng/ml and tacrolimus levels reduced to 3-5 ng/ml.

Retrospective analysis of 67 patients concluded graft survival was similar between reduction and discontinuation of agents. Single center study showed renal allografts were preserved in 8/8 individuals managed with reduction in immunosuppression while graft loss occurred in 8/12 patients treated with an increase in therapy for what was thought to be organ rejection.

Other therapeutic options include Leflunomide, Cidofovir, IVIG, and the fluoroquinolones. Leflunomide is now generally accepted as the second treatment option behind reduction of immunosuppression.

The rationale behind using leflunomide in BKVN comes from its combined immunosuppressive and antiviral properties. Two studies consisting of 26 and 17 patients who developed BKVN on a three-drug regimen of tacrolimus, MMF, and steroids had their MMF replaced with leflunomide 20-60 mg daily. 84 and 88% of patients, respectively had clearance or a progressive reduction in viral load and a stabilization or improvement of graft function (7). [citation needed] In a study conducted by Teschner et al. in 2009, 12/13 patients who had their MMF exchanged with leflunomide cleared the virus by 109 days. In a case series, there was improvement or stabilization in 23/26 patients with BKVN after switching MMF to leflunomide.

There are no dosing guidelines for leflunomide in BKVN. Patient to patient variability has made dosing and monitoring of leflunomide extremely difficult. Study of 26 and 17 patients were dosed between 20 mg/day and 60 mg/day with trough levels of 50-100 mcg/ml. Failure was seen in patients with leflunomide plasma levels <40 mcg/ml. One study of 21 patients found that low levels (<40 mcg/ml) and high levels (>40 mcg/ml) had similar effects on the rate of viral clearance. Those with higher levels had more adverse events (hematologic, hepatic). In the study by Teschner et al., dosages and drug concentration showed no correlation with substantial variation from person to person. In the Teschner study, low drug concentrations were associated with decrease in viral load. This makes it difficult to determine whether or not reduction of viral load or addition of leflunomide was the cause for viral clearance.

Other treatment options include: Quinolone antibiotics: Ciprofloxacin (Cipro) was shown to significantly lower viral loads but no data on survival and graft loss exist; Intravenous immunoglobulin (IVIG) has use in the treatment of infection and allograft rejection—hard to distinguish′ Cidofovir has limited data and is highly nephrotoxic.

A recent study from The Cleveland Clinic reported that BK viremia load >185 000 copies/ml at the time of first positive BKV diagnosis—to be the strongest predictor for BKVAN (97% specificity and 75% sensitivity. In addition the BKV peak viral loads in blood reaching 223 000 copies/ml at any time was found to be predictive for BKVAN (91% specificity and 88% sensitivity (Elfadawy, N S; Flechner, S M; Xiaobo, L; Schold, J; Tian, D; Srinivas, T R; Poggio, E; Fatica, R; Avery, R; Mosaad, S B (2013). “The impact of surveillance and rapid reduction in immunosuppression to control BK virus-related graft injury in kidney transplantation”. Transplant International 26 (8): 822-32).

### JC Virus

The JC virus or John Cunningham virus (JCV) is a type of human polyomavirus (formerly known as papovavirus) and is genetically similar to BK virus and SV40. It was discovered in 1965 by ZuRhein and Chou and by Silverman and Rubinstein and later named using the two initials of a patient with progressive multifocal leukoencephalopathy (PML; BL, Walker D L et al. (1971). “Cultivation of papova-like virus from human brain with progressive multifocal leucoencephalopathy”. Lancet 1 (7712): 1257-60) The virus causes PML and other diseases only in cases of immunodeficiency, as in AIDS or during treatment with drugs intended to induce a state of immunosuppression (e.g., organ transplant patients).

The virus is very common in the general population, infecting 70 to 90 percent of humans; most people acquire JCV in childhood or adolescence (Hansjugen T. Agostini, Caroline F. Ryschkewitsch, Rachel Mory, Elyse J. Singer and Gerald L. Stoner (1997). “JC Virus (JCV) genotypes in brain tissue from patients with progressive multifocal leukoencephalopathy (PML) and in urine from controls without PML: increased frequency of JCV Type 2 in PML”. The Journal of Infectious Diseases (Oxford University Press) 176 (1): 1-8; Laura A. Shackelton, Andrew Rambaut, Oliver G. Pybus, and Edward C. Holmes (2006). “JC Virus evolution and its association with human populations”. Journal of Virology (American Society for Microbiology) 80 (20): 9928-9933; Padgett, B. L. and Walker, D. L. (1973). “Prevalence of antibodies in human sera against JC virus, an isolate from a case of progressive multifocal leukoencephalopathy”. J. Infect. Dis. 127 (4): 467-470). It is found in high concentrations in urban sewage worldwide, leading some researchers to suspect contaminated water as a typical route of infection (Bofill-Mas, S., Formiga-Cruz, M., Clemente-Casares, P., Calafell, F. and Girones, R. (2001). “Potential transmission of human polyomaviruses through the gastrointestinal tract after exposure to virions or viral DNA”. J. Virol. 75 (21): 10290-10299).

The GenBank reference sequence for the JC virus genome is J02226.1. It is noted, however, that minor genetic variations are found consistently in different geographic areas; thus, genetic analysis of JC virus samples has been useful in tracing the history of human migration (Pavesi, A. (2005). “Utility of JC polyomavirus in tracing the pattern of human migrations dating to prehistoric times”. J. Gen. Virol. 86 (Pt 5): 1315-1326). 14 subtypes or genotypes are recognised each associated with a specific geographical region. Three are found in Europe (a, b and c). A minor African type—Af1—occurs in Central and West Africa. The major African type—Af2—is found throughout Africa and also in West and South Asia. Several Asian types are recognised B1-a, B1-b, B1-d, B2, CY, MY and SC. An alternative numbering scheme numbers the genotypes 1-8 with additional lettering. Types 1 and 4 are found in Europe and in indigenous populations in northern Japan, North-East Siberia and northern Canada. These two types are closely related. Types 3 and 6 are found in sub-Saharan Africa: type 3 was isolated in Ethiopia, Tanzania and South Africa. Type 6 is found in Ghana. Both types are also found in the Biaka Pygmies and Bantus from Central Africa. Type 2 has several variants: subtype 2A is found mainly in the Japanese population and native Americans (excluding Inuit); 2B is found in Eurasians; 2D is found in Indians and 2E is found in Australians and western Pacific populations. Subtype 7A is found in southern China and South-East Asia. Subtype 7B is found in northern China, Mongolia and Japan Subtype 7C is found in northern and southern China. Subtype 8 is found in Papua New Guinea and the Pacific Islands.

The initial site of infection may be the tonsils (Monaco, M. C., Jensen, P. N., Hou, J., Durham, L. C. and Major, E. O. (1998). “Detection of JC virus DNA in human tonsil tissue: evidence for site of initial viral infection”. J. Virol. 72 (12): 9918-9923), or possibly the gastrointestinal tract (Bofill-Mas, S., Formiga-Cruz, M., Clemente-Casares, P., Calafell, F. and Girones, R. (2001). “Potential transmission of human polyomaviruses through the gastrointestinal tract after exposure to virions or viral DNA”. J. Virol. 75 (21): 10290-10299). The virus then remains latent in the gastrointestinal tract (Ricciardiello, L., Laghi, L., Ramamirtham, P., Chang, C. L., Chang, D. K., Randolph, A. E. and Boland, C. R. (2000). “JC virus DNA sequences are frequently present in the human upper and lower gastrointestinal tract”. Gastroenterology 119 (5): 1228-1235) and can also infect the tubular epithelial cells in the kidneys (Harvey, R. (2007) Microbiology Philadelphia, Lippincott Williams & Wilkins), where it continues to reproduce, shedding virus particles in the urine.

JCV can cross the blood-brain barrier into the central nervous system, where it infects oligodendrocytes and astrocytes, possibly through the 5-HT2A serotonin receptor (Elphick, G. F., Querbes, W., Jordan, J. A., Gee, G. V., Eash, S., Manley, K., Dugan, A., Stanifer, M., Bhatnagar, A., Kroeze, W. K., Roth, B. L. and Atwood, W. J. (2004). “The human polyomavirus, JCV, uses serotonin receptors to infect cells”. Science 306 (5700): 1380-1383). JC viral DNA can be detected in both non-PML affected and PML-affected (see below) brain tissue (White, F. A., 3rd., Ishaq, M., Stoner, G. L. and Frisque, R. J. (1992). “JC virus DNA is present in many human brain samples from patients without progressive multifocal leukoencephalopathy”. J. Virol. 66 (10): 5726-5734).

Immunodeficiency or immunosuppression allows JCV to reactivate. In the brain it causes the usually fatal progressive multifocal leukoencephalopathy, or PML, by destroying oligodendrocytes. Whether this represents the reactivation of JCV within the CNS or seeding of newly reactivated JCV via blood or lymphatics is unknown. Several studies since 2000 have suggested that the virus is also linked to colorectal cancer, as JCV has been found in malignant colon tumors, but these findings are still controversial (Theodoropoulos, G., Panoussopoulos, D., Papaconstantinou, I., Gazouli, M., Perdiki, M., Bramis, J. and Lazaris, ACh. (2005). “Assessment of JC polyoma virus in colon neoplasms”. Dis. Colon. Rectum. 48 (1): 86-91).

### Determining the Level of Serum Antibody to a Virus

In the context of the present invention, the terms “detect” or “detecting” typically refer to a method that can be used to determine the amount of a protein, or an assessment from which such an amount can be inferred. Examples of such methods include, but are not limited to, Western analysis, ELISA, radioimmunoassay or fluorescence titration assay. Assessing the amount of a marker of viral infection may include assessing the amount of a polypeptide in a sample potentially containing the virus or a marker thereof.

A detection method used in the context of the present invention may include an amplification of the signal caused by the protein, such as the use of the biotin-streptavidin system, for example in form of a conjugation to an immunoglobulin. The detection method may for example include the use of an antibody, e.g. an immunoglobulin, which may be linked to an attached label, such as for instance in Western analysis or ELISA. Where desired, an intracellular immunoglobulin may be used for detection. Some or all of the steps of detection may be part of an automated detection system. As indicated above, the term “antibody” as used herein, is understood to include an immunoglobulin and an immunoglobulin fragment that is capable of specifically binding a selected protein, as well as a respective proteinaceous binding molecule with immunoglobulin-like functions. An antibody may for instance be an EGF-like domain, a Kringle-domain, a fibronectin type I domain, a fibronectin type II domain, a fibronectin type III domain, a PAN domain, a G I a domain, a SRCR domain, a Kunitz Bovine pancreatic trypsin Inhibitor domain, tendamistat. a Kazal-type serine protease inhibitor domain, a Trefoil (P-type) domain, a von Willebrand factor type C domain, an Anaphlatoxin-like domain, a CUB domain, a thyroglobulin type I repeat, an LDL-receptor class A domain, a Sushi domain, a Link domain, a Thrombospondin type I domain, an immunoglobulin domain or a an immunoglobulin-like domain (for example, a domain antibody or a camel heavy chain antibody), a C-type lectin domain, a MAM domain, a von Willebrand factor type A domain, a Somatomedin B domain, a W A P-type four disulfide core domain, a F5/8 type C domain, a Hemopexin domain, an SH2 domain, an SH3 domain, a Lam in in-type EGF-like domain, a C2 domain, a “Kappabody” (Ill. et al. Protein Eng (1997) 10, 949-957), a “Minibody” (Martin et al., EMBO J (1994) 13, 5303-5309), a “Diabody” (Holliger et al., PNAS U.S.A. 90, 6444-6448 (1993)), a “Janusin” (Traunecker et al. EMBO J (1991) 10, 3655-3659 or Traunecker et al., Int J Cancer (1992) Suppl 7, 51-52), a nanobody, an adnectin, a tetranectin, a microbody, an affilin, an affibody or an ankyrin, a crystallin, a knottin, ubiquitin, a zinc-finger protein, an autofluorescent protein, an ankyrin or ankyrin repeat protein or a leucine-rich repeat protein.

A measurement of a level or amount may for instance rely on spectroscopic, photochemical, photometric, fluorometric, radiological, enzymatic or thermodynamic means. An example of a spectroscopical detection method is fluorescence correlation spectroscopy. A photochemical method is for instance photochemical cross-linking. The use of photoactive, fluorescent, radioactive or enzymatic labels respectively are examples for photometric, fluorometric, radiological and enzymatic detection methods. An example of a thermodynamic detection method is isothermal titration calorimetry. As an illustrative example of a label, a detailed protocol on the use of water-soluble, bio-functionalized semiconductor quantum dots has been given by Lidke et al. (Current Protocols in Cell Biology, [2007] Suppl. 36, 25.1.1-25.1.18). Such quantum dots have a particularly high photostability, allowing monitoring their localization for minutes to hours to days. They are typically fluorescent nanoparticles. Since different types of quantum dots can be excited by a single laser line multi-colour labelling can be performed. Detection can for example conveniently be carried out in different fluorescence channels of a flow cytometer.

An immunoglobulin may be monoclonal or polyclonal. The term “polyclonal” refers to immunoglobulins that are heterogenous populations of immunoglobulin molecules derived from the sera of animals immunized with an antigen or an antigenic functional derivative thereof. For the production of polyclonal immunoglobulins, one or more of various host animals may be i minimized by injection with the antigen. Various adjuvants may be used to increase the immunological response, depending on the host species. “Monoclonal immunoglobulins”, also called “monoclonal antibodies”, are substantially homogenous populations of immunoglobulins to a particular antigen. They may be obtained by any technique which provides for the production of immunoglobulin molecules by continuous cell lines in culture. Monoclonal immunoglobulins may be obtained by methods well known to those skilled in the art (see for example, Kohler et al. Nature (1975) 256, 495-497, and U.S. Pat. No. 4,376,110). Routine methods known to those skilled in the art enable production of both immunoglobulins or immunoglobulin fragments and proteinaceous binding molecules with immunoglobulin-like functions, in both prokaryotic and eukaryotic organisms.

In more detail, an immunoglobulin may be isolated by comparing its binding affinity to a protein of interest, with its binding affinity to other polypeptides. Humanized forms of the antibodies of the present invention may be generated using one of the procedures known in the art such as chimerization or CDR grafting. In general, techniques for preparing monoclonal antibodies and hybridomas are well known in the art. Any animal such as a goat, a mouse or a rabbit that is known to produce antibodies can be immunized with the selected polypeptide, e.g. a BK virus coat protein. Methods for immunization are well known in the art. Such methods include subcutaneous or intraperitoneal injection of the polypeptide. One skilled in the art will recognize that the amount of polypeptide used for immunization and the immunization regimen will vary based on the animal which is immunized, including the species of mammal immunized, its immune status and the body weight of the mammal, as well as the antigenicity of the polypeptide and the site of injection.

The polypeptide may be modified or administered in an adjuvant in order to increase the peptide antigenicity. Methods of increasing the antigenicity of a polypeptide are well known in the art. Such procedures include coupling the antigen with a heterologous protein (such as globulin or β-galactosidase) or through the inclusion of an adjuvant during immunization.

For monoclonal immunoglobulins, lymphocytes, typically splenocytes, from the immunized animals are removed, fused with an immortal cell line, typically myeloma cells, and allowed to become monoclonal immunoglobulin producing hybridoma cells. Typically, the immortal cell line such as a myeloma cell line is derived from the same mammalian species as the lymphocytes. Illustrative immortal cell lines are mouse myeloma cell lines that are sensitive to culture medium containing hypoxanthine, aminopterin and thymidine (“HAT medium”). Typically, HAT-sensitive mouse myeloma cells are fused to mouse splenocytes using 1500 molecular weight polyethylene glycol (“PEG 1500”). Hybridoma cells resulting from the fusion may then be selected using HAT medium, which kills unfused and unproductively fused myeloma cells (unfused splenocytes die after several days because they are not transformed).

An ELISA or RIA test can be competitive for measuring the amount of a viral protein and/or a virus-associated protein i.e. a serum antibody to a virus, e.g., BK virus. For example, an enzyme labeled antigen is mixed with a test sample containing antigen, which competes for a limited amount of immunoglobulin or a proteinaceous binding molecule with immunoglobul in-like functions. The reacted (bound) antigen is then separated from the free material, and its enzyme activity is estimated by addition of substrate. An alternative method for antigen measurement is the double immunoglobulin proteinaceous binding molecule sandwich technique. In this modification a solid phase is coated with specific immunoglobulin or a proteinaceous binding molecule with immunoglobulin-like functions. This is then reacted with the sample from the subject that contains the antigen. Then enzyme labeled specific immunoglobulin proteinaceous binding molecule is added, followed by the enzyme substrate. The ‘antigen’ in the test sample is thereby ‘captured’ and immobilized on to the sensitized solid phase where it can itself then immobilize the enzyme labeled immunoglobulin proteinaceous binding molecule. This technique is analogous to the immunoradiometric assays.

In an indirect ELISA method, an antigen is immobilized by passive adsorption on to the solid phase. A test serum may then be incubated with the solid phase and any immunoglobulin in the test serum forms a complex with the antigen on the solid phase. Similarly a solution of a proteinaceous binding molecule with immunoglobulin-like functions may be incubated with the solid phase to allow the formation of a complex between the antigen on the solid phase and the proteinaceous binding molecule. After washing to remove unreacted serum components an immunoglobulin or proteinaceous binding molecule with immunoglobulin-like functions, linked to an enzyme is contacted with the solid phase and incubated. Where the second reagent is selected to be a proteinaceous binding molecule with immunoglobulin-like functions, a respective proteinaceous binding molecule that specifically binds to the proteinaceous binding molecule or the immunoglobulin directed against the antigen is used. A complex of the second proteinaceous binding molecule or immunoglobulin and the first proteinaceous binding molecule or immunoglobulin, bound to the antigen, is formed. Washing again removes unreacted material. In the case of RIA, radioactivity signals are being detected. In the case of ELISA the enzyme substrate is added. Its color change will be a measure of the amount of the immobilized complex involving the antigen, which is proportional to the antibody level in the test sample.

In another embodiment the immunoglobulin or the proteinaceous binding molecule with immunoglobulin-like functions may be immobilized onto a surface, such as the surface of a polymer bead, or coated onto the surface of a device such as a polymer plate or a glass plate. As a result the immune complexes can easily be separated from other components present by simply washing the surface, e.g. the beads or plate. This is the most common method currently used in the art and is referred to as solid phase RIA or ELISA. This embodiment may be particularly useful for determining the amount of viral protein and/or serum antibody to a virus. On a general basis, in any embodiment of a radiolabel assay or of an enzyme-immunoassay passive adsorption to the solid phase can be used in the first step. Adsorption of other reagents can be prevented by inclusion of wetting agents in all the subsequent washing and incubation steps. It may be advantageous to perform washing to prevent carry-over of reagents from one step to the next.

Various other modifications of ELISA have been used in the art. For example, a system where the second proteinaceous binding molecule or immunoglobulin used in the double antibody sandwich method is from a different species, and this is then reacted with an anti-immunoglobulin enzyme conjugate or an anti-proteinaceous binding molecule enzyme conjugate.

This technique comes with the potential advantage that it avoids the labeling of the specific immunoglobulin or proteinaceous binding molecule, which may be in short supply and of low potency. This same technique can be used to assay immunoglobulin or proteinaceous binding molecule where only an impure antigen is available; the specific reactive antigens are selected by the antibody immobilized on the solid phase.

In another example of an ELISA assay for an antigen, a surface, a specific antigen is immobilized on a surface, e.g. a plate used, and the surface is then incubated with a mixture of reference immunoglobulins or proteinaceous binding molecules and a test sample, if there is no antigen in the test sample the reference immunoglobulin or proteinaceous binding molecule becomes fixed to an antigen sensitized surface, if there is antigen in the test solution this combines with the reference immunoglobulin or proteinaceous binding molecule, which cannot then react with the sensitized solid phase. The amount of immunoglobulin proteinaceous binding molecule attached is then indicated by an enzyme labeled anti-globulin, anti-binding molecule conjugate and enzyme substrate. The amount of inhibition of substrate degradation in the test sample (as compared with the reference system) is proportional to the amount of antigen in the test system.

### Pharmaceutical Compositions

In certain embodiments, the present invention provides for a pharmaceutical composition comprising the humanized monoclonal antibody targeting immune function employed in the present invention. The humanized monoclonal antibody targeting immune function can be suitably formulated and introduced into a subject or the environment of the cell by any means recognized for such delivery.

Such compositions typically include the agent and a pharmaceutically acceptable carrier. As used herein the language “pharmaceutically acceptable carrier” includes saline, solvents, dispersion media, coatings, antibacterial and antifungal agents, isotonic and absorption delaying agents, and the like, compatible with pharmaceutical administration. Supplementary active compounds can also be incorporated into the compositions.

A pharmaceutical composition is formulated to be compatible with its intended route of administration. Examples of routes of administration include parenteral, e.g., intravenous, intradermal, subcutaneous, oral (e.g., inhalation), transdermal (topical), transmucosal, and rectal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In all cases, the composition must be sterile and should be fluid to the extent that easy syringability exists. It should be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyetheylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as manitol, sorbitol, sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound in the required amount in a selected solvent with one or a combination of ingredients enumerated above, as required, followed by filtered sterilization. Generally, dispersions are prepared by incorporating the active compound into a sterile vehicle, which contains a basic dispersion medium and the required other ingredients from those enumerated above. In the case of sterile powders for the preparation of sterile injectable solutions, the preferred methods of preparation are vacuum drying and freeze-drying which yields a powder of the active ingredient plus any additional desired ingredient from a previously sterile-filtered solution thereof.

Oral compositions generally include an inert diluent or an edible carrier. For the purpose of oral therapeutic administration, the active compound can be incorporated with excipients and used in the form of tablets, troches, or capsules, e.g., gelatin capsules. Oral compositions can also be prepared using a fluid carrier for use as a mouthwash. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the compounds are delivered in the form of an aerosol spray from pressured container or dispenser which contains a suitable propellant, e.g., a gas such as carbon dioxide, or a nebulizer. Such methods include those described in U.S. Pat. No. 6,468,798.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

The compounds can also be prepared in the form of suppositories (e.g., with conventional suppository bases such as cocoa butter and other glycerides) or retention enemas for rectal delivery.

In one embodiment, the active compounds are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Such formulations can be prepared using standard techniques. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

Toxicity and therapeutic efficacy of such compounds can be determined by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effects is the therapeutic index and it can be expressed as the ratio LD50/ED50. Compounds which exhibit high therapeutic indices are preferred. While compounds that exhibit toxic side effects may be used, care should be taken to design a delivery system that targets such compounds to the site of affected tissue in order to minimize potential damage to uninfected cells and, thereby, reduce side effects.

The data obtained from cell culture assays and animal studies can be used in formulating a range of dosage for use in humans. The dosage of such compounds lies preferably within a range of circulating concentrations that include the ED50 with little or no toxicity. The dosage may vary within this range depending upon the dosage form employed and the route of administration utilized. For a compound used in the method of the invention, the therapeutically effective dose can be estimated initially from cell culture assays. A dose may be formulated in animal models to achieve a circulating plasma concentration range that includes the IC50 (i.e., the concentration of the test compound which achieves a half-maximal inhibition of symptoms) as determined in cell culture. Such information can be used to more accurately determine useful doses in humans. Levels in plasma may be measured, for example, by high performance liquid chromatography.

As defined herein, a therapeutically effective amount of a humanized monoclonal antibody targeting immune function (i.e., an effective dosage) depends on the humanized monoclonal antibody selected. For instance, single dose amounts of a humanized monoclonal antibody targeting immune function in the range of approximately 1 pg to 1000 mg may be administered; in some embodiments, 10, 30, 100, or 1000 pg, or 10, 30, 100, or 1000 ng, or 10, 30, 100, or 1000 μg, or 10, 30, 100, or 1000 mg may be administered. In some embodiments, 1-5 g of the compositions can be administered. The compositions can be administered one from one or more times per day to one or more times per week; including once every other day. The skilled artisan will appreciate that certain factors may influence the dosage and timing required to effectively treat a subject, including but not limited to the severity of the disease or disorder, previous treatments, the general health and/or age of the subject, and other diseases present. Moreover, treatment of a subject with a therapeutically effective amount of a humanized monoclonal antibody targeting immune function can include a single treatment or, preferably, can include a series of treatments.

It can be appreciated that the method of introducing a humanized monoclonal antibody targeting immune function into the environment of a cell will depend on the type of cell and the make up of its environment.

Suitable amounts of humanized monoclonal antibody or antibodies targeting immune function must be introduced and these amounts can be empirically determined using standard methods. Exemplary effective concentrations of individual humanized monoclonal antibody species in the environment of a cell can be 500 millimolar or less, 50 millimolar or less, 10 millimolar or less, 1 millimolar or less, 500 nanomolar or less, 50 nanomolar or less, 10 nanomolar or less, or even compositions in which concentrations of 1 nanomolar or less can be used.

The pharmaceutical compositions can be included in a kit, container, pack, or dispenser together with instructions for administration.

### Detection Methods

Numerous methods and devices are well known to the skilled artisan for the detection and analysis of serum antibody to BK and/or JC virus) of the instant invention.

With regard to polypeptides or proteins in patient test samples, immunoassay devices and methods can be used. See, e.g., U.S. Pat. Nos. 6,143,576; 6,113,855; 6,019,944; 5,985,579; 5,947,124; 5,939,272; 5,922,615; 5,885,527; 5,851,776; 5,824,799; 5,679,526; 5,525,524; and 5,480,792, each of which is hereby incorporated by reference in its entirety, including all tables, figures and claims. These devices and methods can utilize labeled molecules in various sandwich, competitive, or non-competitive assay formats, to generate a signal that is related to the presence or amount of a polypeptide of interest.

Additionally, certain methods and devices, such as biosensors and optical immunoassays, may be employed to determine the presence or amount of polypeptides without the need for a labeled molecule. See, e.g., U.S. Pat. Nos. 5,631,171; and 5,955,377, each of which is hereby incorporated by reference in its entirety, including all tables, figures and claims. One skilled in the art also recognizes that robotic instrumentation including but not limited to Beckman ACCESS®, Abbott AXSYM®, Roche ELECSYS®, Dade Behring STRATUS® systems are among the immunoassay analyzers that are capable of performing the immunoassays taught herein.

In certain embodiments, the markers of viral infection are analyzed using an immunoassay, although other methods are well known to those skilled in the art. The presence or amount of a marker is generally determined using antibodies specific for each marker and detecting specific binding. Any suitable immunoassay may be utilized, for example, enzyme-linked immunoassays (ELISA), radioimmunoassays (RIAs), competitive binding assays, and the like. Specific immunological binding of the antibody to the marker can be detected directly or indirectly. Direct labels include fluorescent or luminescent tags, metals, dyes, radionuclides, and the like, attached to the antibody. Indirect labels include various enzymes well known in the art, such as alkaline phosphatase, horseradish peroxidase and the like.

The use of immobilized antibodies specific for the markers is also contemplated by the present invention. The antibodies could be immobilized onto a variety of solid supports, such as magnetic or chromatographic matrix particles, the surface of an assay place (such as microtiter wells), pieces of a solid substrate material or membrane (such as plastic, nylon, paper), and the like. An assay strip could be prepared by coating the antibody or a plurality of antibodies in an array on solid support. This strip could then be dipped into the test sample and then processed quickly through washes and detection steps to generate a measurable signal, such as a colored spot.

The analysis of a plurality of markers may be carried out separately or simultaneously with one test sample. For separate or sequential assay of markers, suitable apparatuses include clinical laboratory analyzers such as the ELECSYS® (Roche), the AXSYM® (Abbott), the ACCESS® (Beckman), the ADVIA® CENTAUR® (Bayer) immunoassay systems, the NICHOLS ADVANTAGE® (Nichols Institute) immunoassay system, etc. Preferred apparatuses or protein chips perform simultaneous assays of a plurality of markers on a single surface. Particularly useful physical formats comprise surfaces having a plurality of discrete, addressable locations for the detection of a plurality of different analytes. Such formats include protein microarrays, or “protein chips” (see, e.g., Ng and Ilag, J. Cell Mol. Med. 6: 329-340 (2002)) and certain capillary devices (see e.g., U.S. Pat. No. 6,019,944). In these embodiments each discrete surface location may comprise antibodies to immobilize one or more analyte(s) (e.g., a marker) for detection at each location. Surfaces may alternatively comprise one or more discrete particles (e.g., microparticles or nanoparticles) immobilized at discrete locations of a surface, where the microparticles comprise antibodies to immobilize one analyte (e.g., a marker) for detection. As noted, many protein biochips are described in the art. These further include, for example, protein biochips produced by Ciphergen Biosystems, Inc. (Fremont, Calif.), Packard BioScience Company (Meriden Conn.), Zyomyx (Hayward, Calif.), Phylos (Lexington, Mass.) and Biacore (Uppsala, Sweden). Examples of such protein biochips are described in the following patents or published patent applications: U.S. Pat. No. 6,225,047; PCT International Publication No. WO 99/51773; U.S. Pat. No. 6,329,209, PCT International Publication No. WO 00/56934 and U.S. Pat. No. 5,242,828, each of which are incorporated by reference.

Several markers may be combined into one test for efficient processing of a multiple of samples. In addition, one skilled in the art would recognize the value of testing multiple samples (for example, at successive time points) from the same individual. Such testing of serial samples will allow the identification of changes in marker levels over time. Increases or decreases in marker levels, as well as the absence of change in marker levels, would provide useful information about the disease/infection status that includes, but is not limited to identifying the approximate time from onset of the event/infection, the appropriateness of drug therapies, the effectiveness of various therapies, identification of the disease severity, and identification of the patient's outcome, including risk of future events.

### Kits

In another embodiment, the invention provides kits for assessing the risk of PML in a patient. Depending on how the kit is to be operated, the kit may include one or more antibodies or other binding moieties that specifically bind to any of the marker polypeptides of the virus-targeted serum antibodies of the invention. If the kit is to be used to detect antibody molecules that target the virus(es) of the invention, the kit may include specific antibodies or antibody binding agent(s) (optionally, labeled forms of such) for use in the detection of the marker antibody in a sample (e.g., via ELISA or other binding assay). Both antibody and antigen preparations should preferably be provided in a suitable titrated form, with antigen concentrations and/or antibody titers given for easy reference in quantitative applications.

In certain embodiments, the kits may include an immunodetection reagent or label for the detection of specific immunoreaction between markers and/or antibody, as the case may be, and the diagnostic sample. Suitable detection reagents are well known in the art as exemplified by radioactive, enzymatic or otherwise chromogenic ligands, which are typically employed in association with the antigen and/or antibody, or in association with a second antibody having specificity for first antibody. Thus, the reaction is detected or quantified by means of detecting or quantifying the label Immunodetection reagents and processes suitable for application in connection with the novel methods of the present invention are generally well known in the art.

The reagents may also include ancillary agents such as buffering agents and protein stabilizing agents, e.g., polysaccharides and the like. The diagnostic kit may further include where necessary agents for reducing background interference in a test, agents for increasing signal, apparatus for conducting a test, calibration curves and charts, standardization curves and charts, and the like.

The kit can also comprise a washing solution or instructions for making a washing solution, in which the combination of the capture reagent and the washing solution allows capture of the marker or markers on the solid support for subsequent detection by, e.g., mass spectrometry. The kit may include more than type of adsorbent, each present on a different solid support.

In a further embodiment, such a kit can comprise instructions for suitable operational parameters in the form of a label or separate insert. For example, the instructions may inform a consumer about how to collect the sample, how to wash the probe or the particular markers to be detected.

In yet another embodiment, the kit can comprise one or more containers with marker samples, to be used as standard(s) for calibration.

The probes of the invention can also be proteinaceous materials, e.g., polypeptides or polypeptide fragments of the markers of the virus(es) of the invention. In another embodiment, the probe may be a proteinaceous compound. There are a wide variety of protein-protein interactions; however, proteins also bind nucleic acids, metals and other non-proteinaceous compounds (e.g., lipids, hormones, transmitters). Some other examples of protein that may be used as either targets or probes include, but are not limited to, antibodies, enzymes, receptors, and DNA- or RNA-binding proteins.

In various embodiments, it may desirable to label probe or target molecules. Examples of labels include paramagnetic ions, radioactive isotopes; fluorochromes, NMR-detectable substances, and X-ray imaging compounds.

Paramagnetic ions include chromium (III), manganese (II), iron (III), iron (II), cobalt (II), nickel (II), copper (II), neodymium (II), samarium (III), ytterbium (III), gadolinium (III), vanadium (II), terbium (III), dysprosium (III), holmium (III) and/or erbium (III), with gadolinium being particularly preferred. Ions useful in other contexts, such as X-ray imaging, include but are not limited to lanthanum (III), gold (III), lead (II), and especially bismuth (III).

Radioactive isotopes include ¹⁴carbon, ¹⁵chromium, ³⁶chlorine, ⁵⁷cobalt, and the like may be utilized. Among the fluorescent labels contemplated for use include Alexa 350, Alexa 430, AMCA, BODIPY 630/650, BODIPY 650/665, BODIPY-FL, BODIPY-R6G, BODIPY-TMR, BODIPY-TRX, Cascade Blue, Cy3, Cy5,6-FAM, Fluorescein Isothiocyanate, HEX, 6-JOE, Oregon Green 488, Oregon Green 500, Oregon Green 514, Pacific Blue, REG, Rhodamine Green, Rhodamine Red, Renographin, ROX, TAMRA, TET, Tetramethylrhodamine, and/or Texas Red. Enzymes (an enzyme tag) that will generate a colored product upon contact with a chromogenic substrate may also be used. Examples of suitable enzymes include urease, alkaline phosphatase, (horseradish) hydrogen peroxidase or glucose oxidase. Preferred secondary binding ligands are biotin and/or avidin and streptavidin compounds. The use of such labels is well known to those of skill in the art and are described, for example, in U.S. Pat. Nos. 3,817,837; 3,850,752; 3,939,350; 3,996,345; 4,277,437; 4,275,149 and 4,366,241; each incorporated herein by reference.

### Disease Targets of Monoclonal Antibody Therapies

Multiple sclerosis (MS) is a chronic, inflammatory central nervous system (CNS) disease, characterized pathologically by demyelination. MS has also been classified as an autoimmune disease. MS disease activity can be monitored by cranial scans, including magnetic resonance imaging (MRI) of the brain, accumulation of disability, as well as rate and severity of relapses. There are five distinct disease stages and/or types of MS, namely. (1) benign multiple sclerosis; (2) relapsing-remitting multiple sclerosis; (3) secondary progressive multiple sclerosis; (4) progressive relapsing multiple sclerosis; and (5) primary progressive multiple sclerosis.

Crohn's disease is a type of inflammatory bowel disease. It typically manifests in the gastrointestinal tract and can be categorized by the specific tract region affected. It is thought to be an autoimmune disease, in which the body's immune system attacks the gastrointestinal tract, causing inflammation of the gastrointestinal tract. The disease manifestations usually are isolated to the digestive tract, but other manifestations such as inflammation of skin structures, the eyes, and the joints have been well described. The disease is known to have spontaneous exacerbations and remissions. Unfortunately, the cause of Crohn's disease is not known, and there is no known cure for Crohn's disease.

Crohn's disease has an immune response pattern that includes an increased production of interleukin-12, tumour necrosis factor (TNF) and interferon-γ. Tumor necrosis factor (TNF) has been identified as an important cytokine in the pathogenesis of Crohn's disease, with elevated concentrations playing a role in pathologic inflammation. The increased production of TNF by macrophages in patients with Crohn's disease results in elevated concentrations of TNF in the stool, blood, and mucosa, in recent years, biologic response modifiers that inhibit TNF activity have become potential therapies for treating Crohn's disease.

The humanized monoclonal immunoglobulin Natalizumab, directed against the α₄-subunit of α₄β₁-integrin (VLA-4, Very Late Antigen-4) and α₄β₇ (LPAM-1, Lymphocyte Payer's Patch Adhesion Molecule 1) integrins expressed on the surface of activated lymphocytes, has been used in the treatment of both MS and Crohn's disease, and is also being tested for clinical efficacy against neoplastic conditions. Natalizumab is both clinically effective and generally well-tolerated. However, Natalizumab treatment for longer than 18 months has been found to be associated with an enhanced risk of developing PML. PML has almost exclusively been found in immunocompromised individuals, especially in subjects with reduced cellular immunity. It has also been reported in rheumatic diseases. PML has for example been found in individuals with hematological malignancies and lymphoproliferative diseases, individuals with Hodgkin's lymphoma, individuals with systemic lupus erythematosus or subjects receiving immunosuppressive medication, such as transplant patients. In addition to Natalizumab therapy, PML has also been found to be associated with therapy using the monoclonal antibodies Rituximab, used in the treatment of lymphomas, leukemias, transplant rejection and certain autoimmune disorders, and Efalizumab, formerly used in the treatment of autoimmune diseases, in particular, psoriasis. In view of the risk of PML, Efalizumab has currently been withdrawn from the U.S. market. Natalizumab, first approved in 2004 by the U.S. Food and Drug Administration (FDA) for the treatment of multiple sclerosis, was withdrawn from the market after it was linked with three cases of PML. After a review of safety information and no additional cases of PML were identified in previously treated patients, the antibody was re-introduced in the U.S. and approved in the European Union in July 2006. Natalizumab has now been restricted as a monotherapy for adult relapsing remitting multiple sclerosis (RRMS) patients with high disease activity. Natalizumab is also still approved as a monotherapy for adults with moderate-to-severe active Crohn's disease.

PML is caused by lytic infection of oligodendrocytes by the John Cunningham virus (JCV), a double-stranded, not enveloped human polyomavirus. As of Aug. 5, 2014, there have been 486 confirmed cases of PML worldwide among 129,100 patients treated with Natalizumab (data from the Biogen Idec website). It is still largely unknown how the treatment with blocking integrins α₄β₁/VLA-4 and/or α₄β₇/LPAM-1 interferes with JCV control or immune surveillance (Tan, C. S, and Koralnik, U., Lancet Neurol. (2010) 9, 4, 425-437). The majority of PML cases are, nevertheless, represented by individuals infected with HIV. While the availability of potent antiretroviral therapies has led to a decrease in the incidence of PML, HIV/AIDS-associated PML morbidity and mortality remain high (Hernandez, B., et al. Expert Opin. Pharmacother. [2009] 10, 3, 403-416). JCV is difficult to study, as it grows only in a few cell types in vitro (human fetal glial cells or adult glioma or neuroblastoma cell lines) and no animal models exist.

Prognosis of PML is poor, since no specific therapy is available. While only 20% of the Natalizumab-treated PML patients so far have died, the overall mortality of PML has been reported to be above 50% attributable to the disease. In the absence of any therapy, it would be particularly helpful to be able to predict the risk whether an individual, in particular an individual suffering from a condition or disease for which natalizumab or other monoclonal antibody or other therapy known to run a risk of triggering PML, is likely to develop PML. Hence, there exists a need for means to determine at an early stage, i.e. during initial diagnosis of a disease or condition potentially treatable with natalizumab or other monoclonal antibody or other therapy known to run a risk of triggering PML, or even before the onset of such disease or condition, whether such an individual is likely to suffer from PML.

Recent studies suggest that patients under treatment with the α₄-integrin-blocking agent Natalizumab for more than 12 months are at elevated risk for PML, with the risk increasing after approximately 18 months of treatment, and can reach risk levels of up to 1:120. It is not known if the risk of developing PML continues to increase, remains the same, or decreases after a patient has been on Natalizumab for more than three years. Since there is a clear risk association between Natalizumab and the development of PML after long-term treatment of the α₄-integrin-blocking agent Natalizumab, there is an urgent need to identify those patients who are more prone to PML. However, only few candidates as indicators in this regard have evolved prior to the instant invention: (1) treatment duration, (2) pre-treatment with immunosuppressive drugs, and (3) presence of JCV antibodies in serum. Thus, the current invention provides an additional critical option for assessing the likelihood of PML development in a subject.

European patent application EP 2226392 A1 discloses an immunological method for detecting an extra renal active infection by JCV in a patient who is a candidate for immunosuppressive treatment. The method of EP 2226392 A1 includes screening for the presence of activated T lymphocytes against JCV.

U.S. patent application 2010/0196318 discloses testing for serum anti-JCV antibody prior to initiating Natalizumab therapy in patients. However, the detection of JCV antibody in an individual does not predict the risk for PML and therefore cannot advise a medical professional whether or not to continue the treatment. U.S. patent application 2009/021107 discloses a method of screening patients undergoing Natalizumab treatment by testing the patient's cerebrospinal fluid to detect the presence of cytomegalovirus, JCV, Toxoplasma gondii, Epstein-Barr virus, Cryptococcus neoformans and tuberculosis by PGR, as well as examining the retinal status to detect the presence of ocular cytomegalovirus. If an indication of the presence of the virus is detected, Natalizumab treatment should be discontinued. However, such methods are only precautionary measures which also do not indicate a risk of developing PML. There still remains a need to develop a method to determine the risk of a subject to develop PML who receive an α₄-integrin-blocking agent on an individual basis. It would be advantageous if the determination could help the practitioner to identify patients who are particularly prone to PML or stop the treatment in time before the immune competence of the subject deteriorates.

It is therefore an object of the present invention to provide a method that is suitable for determining the risk for PML development in a subject. It would be advantageous if such method can be used to monitor the immune competence of patients receiving or expected to receive Natalizumab thus to avoid the possible development of PML or even another complication at a later stage. These objects are solved by the methods of the independent claims.

### Dosage

Dosage of one or more agents of the invention (e.g., a humanized antibody targeting immune function) can be determined by one of skill in the art and can also be adjusted by the individual physician in the event of any complication. Typically, the dosage ranges from 0.001 mg/kg body weight to 5 g/kg body weight. In some embodiments, the dosage range is from 0.001 mg/kg body weight to 1 g kg body weight, from 0.001 mg kg body weight to 0.5 g/kg body weight, from 0.001 mg/kg body weight to 0.1 g/kg body weight, from 0.001 mg/kg body weight to 50 mg/kg body weight, from 0.001 mg/kg body weight to 25 mg/kg body weight, from 0.001 mg/kg body weight to 10 mg/kg body weight, from 0.001 mg/kg body weight to 5 mg/kg body weight, from 0.001 mg/kg body weight to 1 mg/kg body weight, from 0.001 mg/kg body weight to 0.1 mg/kg body weight, from 0.001 mg/kg body weight to 0.005 mg/kg body weight. Alternatively, in some embodiments the dosage range is from 0.1 g/kg body weight to 5 g/kg body weight, from 0.5 g/kg body weight to 5 g/kg body weight, from 1 g/kg body weight to 5 g/kg body weight, from 1.5 g/kg body weight to 5 g/kg body weight, from 2 g/kg body weight to 5 g/kg body weight, from 2.5 g/kg body weight to 5 g/kg body weight, from 3 g/kg body weight to 5 g/kg body weight, from 3.5 g/kg body weight to 5 g/kg body weight, from 4 g/kg body weight to 5 g/kg body weight, from 4.5 g/kg body weight to 5 g/kg body weight, from 4.8 g/kg body weight to 5 g/kg body weight. In one embodiment, the dose range is from 5 g/kg body weight to 30 g/kg body weight. Alternatively, the dose range will be titrated to maintain serum levels between 5 g/mL and 30 g/mL.

Administration of the doses recited above can be repeated for a limited period of time. In some embodiments, the doses are given once a day, or multiple times a day, for example but not limited to three times a day. In one embodiment, the doses recited above are administered daily for several weeks or months. The duration of treatment depends upon the subject's clinical progress and responsiveness to therapy. Continuous, relatively low maintenance doses are contemplated after an initial higher therapeutic dose.

### Methods of Treatment

The present invention provides for both prophylactic and therapeutic methods of identifying (detecting) and treating a subject at risk of (or susceptible to) PML in treating an underlying immune/autoimmune condition or disorder, or a neoplastic disease or disorder. “Treatment”, or “treating” as used herein, is defined as the application or administration of a therapeutic agent (e.g., a humanized monoclonal antibody targeting immune function) to a patient, or application or administration of a therapeutic agent to an isolated tissue or cell line from a patient, who has the disease or disorder, a symptom of disease or disorder or a predisposition toward a disease or disorder, with the purpose to cure, heal, alleviate, relieve, alter, remedy, ameliorate, improve or affect the disease or disorder, the symptoms of the disease or disorder, or the predisposition toward disease.

In one aspect, the invention provides a method for preventing in a subject, a disease or disorder as described above, by excluding or discontinuing the subject from administration of a therapeutic agent (e.g., a humanized monoclonal antibody targeting immune function). Subjects at risk for a disease (or at reduced risk for developing a disease, e.g., PML) can be identified by, for example, one or a combination of diagnostic or prognostic assays as described herein.

With regards to both prophylactic and therapeutic methods of treatment, such treatments may be specifically tailored or modified, based on knowledge obtained from the field of pharmacogenomics. “Pharmacogenomics”, as used herein, refers to the application of genomics technologies such as gene sequencing, statistical genetics, and gene expression analysis to drugs in clinical development and on the market. More specifically, the term refers the study of how a patient's genes determine his or her response to a drug (e.g., a patient's “drug response phenotype”, or “drug response genotype”). Thus, another aspect of the invention provides methods for tailoring an individual's prophylactic or therapeutic treatment according to that individual's drug response genotype, expression profile, biomarkers, etc. Pharmacogenomics allows a clinician or physician to target prophylactic or therapeutic treatments to patients who will most benefit from the treatment and to avoid treatment of patients who will experience toxic drug-related side effects.

The practice of the present invention employs, unless otherwise indicated, conventional techniques of chemistry, molecular biology, microbiology, recombinant DNA, genetics, immunology, cell biology, cell culture and transgenic biology, which are within the skill of the art. See, e.g., Maniatis et al., 1982, Molecular Cloning (Cold Spring Harbor Laboratory Press, Cold Spring Harbor, N.Y.); Sambrook et al., 1989, Molecular Cloning, 2nd Ed. (Cold Spring Harbor Laboratory Press, Cold Spring Harbor, N.Y.); Sambrook and Russell, 2001, Molecular Cloning, 3rd Ed. (Cold Spring Harbor Laboratory Press, Cold Spring Harbor, N.Y.); Ausubel et al., 1992), Current Protocols in Molecular Biology (John Wiley & Sons, including periodic updates); Glover, 1985, DNA Cloning (IRL Press, Oxford); Anand, 1992; Guthrie and Fink, 1991; Harlow and Lane, 1988, Antibodies, (Cold Spring Harbor Laboratory Press, Cold Spring Harbor, N.Y.); Jakoby and Pastan, 1979; Nucleic Acid Hybridization (B. D. Hames & S. J. Higgins eds. 1984); Transcription And Translation (B. D. Hames & S. J. Higgins eds. 1984); Culture Of Animal Cells (R. I. Freshney, Alan R. Liss, Inc., 1987); Immobilized Cells And Enzymes (IRL Press, 1986); B. Perbal, A Practical Guide To Molecular Cloning (1984); the treatise, Methods In Enzymology (Academic Press, Inc., N.Y.); Gene Transfer Vectors For Mammalian Cells (J. H. Miller and M. P. Calos eds., 1987, Cold Spring Harbor Laboratory); Methods In Enzymology, Vols. 154 and 155 (Wu et al. eds.), Immunochemical Methods In Cell And Molecular Biology (Mayer and Walker, eds., Academic Press, London, 1987); Handbook Of Experimental Immunology, Volumes I-IV (D. M. Weir and C. C. Blackwell, eds., 1986); Riott, Essential Immunology, 6th Edition, Blackwell Scientific Publications, Oxford, 1988; Hogan et al., Manipulating the Mouse Embryo, (Cold Spring Harbor Laboratory Press, Cold Spring Harbor, N. Y., 1986); Westerfield, M., The zebrafish book. A guide for the laboratory use of zebrafish (Danio rerio), (4th Ed., Univ. of Oregon Press, Eugene, 2000).

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

### EXAMPLES

The present invention is described by reference to the following Examples, which are offered by way of illustration and are not intended to limit the invention in any manner. Standard techniques well known in the art or the techniques specifically described below were utilized.

### Example 1

### BK Virus Serology for Risk Stratification of Progressive Multifocal Leukoencephalopathy in Patients Treated with Natalizumab (Tysabri)

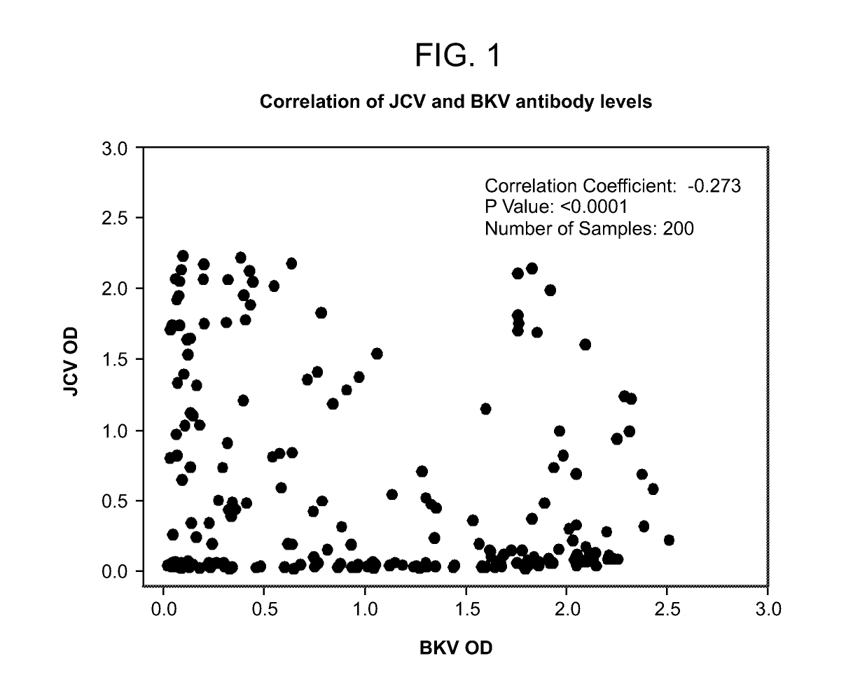

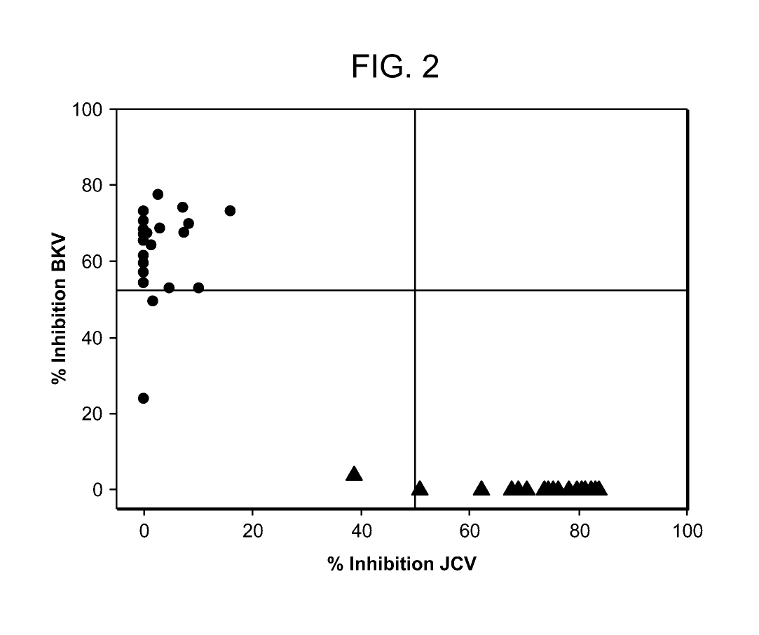

Serological measures of exposure to a closely JCV-related human polyomavirus, BK virus, were assessed for the ability to more precisely define those individuals at risk of PML. In detecting polyomavirus infection in subjects, it has been observed that the level of antibody to JC virus was inversely correlated to the level of antibody to BK virus (FIG. 1; Major, 2001). Because the risk of PML has been described as related to the level of antibody to JC virus and the level of BK antibody was observed to be inversely related to JC antibody levels, it was hypothesized that a low level of antibody to BK would be associated with a risk of PML. Without wishing to be bound by theory, the mechanistic basis for a high risk of PML in individuals with low levels of antibody to BK virus is believed to be related to the pathophysiology of polyomavirus infections and the nature of immunological cross-reactivity between the viruses. Specifically, it was shown that there was no serological cross-reactivity between JC and BK virus (FIG. 2; Viscidi et al., 2011; Viscidi and Clayman, 2006), and thus no cross-protective neutralizing antibodies that would prevent acquisition of infection. However, the JC and BK viruses share 80-85% similarity in amino acid sequence, and cross-reactive T cell epitopes have been mapped in the VP1 gene of the two viruses (Li et al., 2006; Sharma et al., 2006).

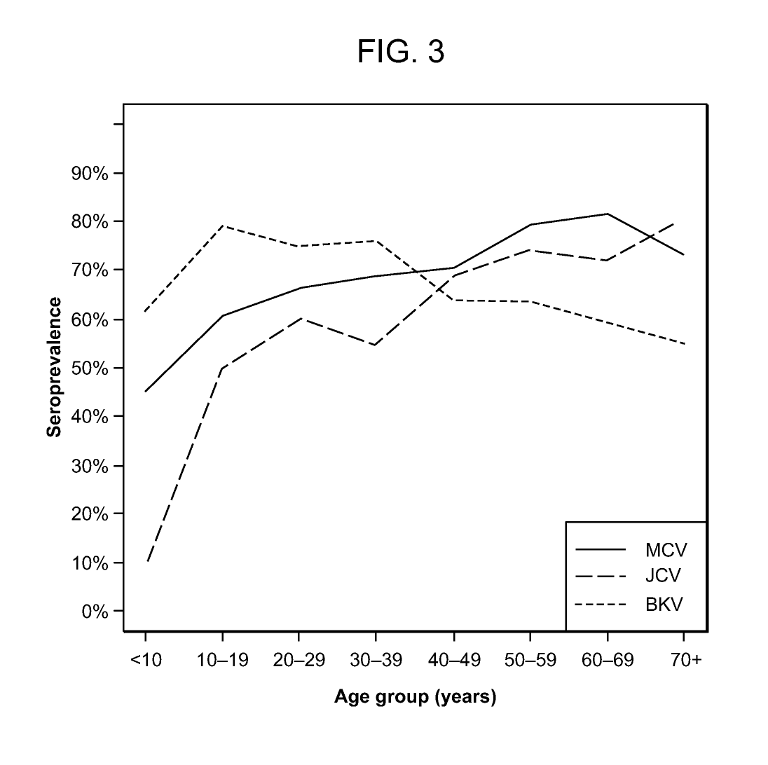

It was hypothesized that in the absence of prior immunity to BK virus, reflected by a low antibody level, a person infected with JC virus would experience a more widespread viral infection, with higher viral load and induction of higher antibody levels. These individuals would be at risk of PML. However, the proportion of individuals at risk of PML in the population was very small and it was determined that this was explained by the epidemiology of BK and JC virus infection. In seroepidemiological studies, it was shown that exposure to BK virus occurred very early in life and generally before exposure to JC virus (FIG. 3; Viscidi et al., 2011; Knowles et al., 2003). Thus, the great majority of individuals would have some degree of T cell cross protective immunity at the time of exposure to JC virus. While this would not protect them from acquiring a JC infection, it would limit the extent of the infection and thus reduce their risk of PML later in life.

### Example 2

### Risk Prediction of PML in Patients with Human Immunodeficiency Virus Infection

A nested case-control study was performed using archived samples from the MACS, a prospective study of HIV/AIDS in homosexual men in the United States begun in 1984. Demographic and clinical data and serum were collected every 6 months for 2.5 years prior to the diagnosis of progressive mutifocal leukoencephalopathy (PML). Twenty five PML patients from MACS were diagnosed between 1985 and 1996. Cases were matched with 80 HIV-seropositive participants, who did not develop PML. A virus-like particle (VLP)-based enzyme-linked immunosorbent assay (ELISA) was used to detect serum antibody to JC and BK genotype I capsids, as described previously. To construct a VLP for a BK genotype IV strain, the amino acid sequence of BK IV strain MMR-29 VP1 protein (Genbank accession number AB269844) was converted in silico to a nucleotide sequence corresponding to the preferred codon usage of Drosophila melanogaster. The nucleotide sequence of BK virus strain MMR-29 having genotype IVc1 VP1 gene with Drosophila melanogaster high codon usage is:

5′-ATGGCCCCCACTAAGCGTAAGGGTGAGTGCCCAGGCGCTGCCCCC
AAGAAGCCCAAGGAGCCGGTGCAGGTCCCCAAGCTGCTGATTAAGGGC
GGAGTGGAGGTGCTGGAGGTCAAGACCGGAGTCGATGCCATCACCGAG
GTCGAGTGCTTCCTGAATCCCGAGATGGGAGACCCCGACAACGACCTG
CGCGGCTACTCCTTGCGCCTGACCGCCGAGACCGCCTTCGACAGCGAT
TCGCCCGACCGCAAGATGCTGCCGTGCTACTCCACCGCCCGTATCCCC
CTGCCAAACCTCAACGAGGATCTGACCTGCGGCAACCTGCTGATGTGG
GAGGCCGTCACCGTGAAGACCGAGGTGATCGGAATTACTTCCATGCTG
AACCTGCACGCCGGATCCCAGAAGGTTCACGAGAACGGCGGCGGCAAG
CCCGTCCAGGGATCCAACTTCCACTTCTTCGCCGTCGGAGGCGATCCC
CTGGAAATGCAGGGTGTGTTGATGAACTATCGCACCAAGTACCCCGAG
GGCACTGTGACCCCCAAGAACCCCACCGCCCAGAGCCAGGTGATGAAC
ACCGACCATAAGGCCTACTTGGATAAGAATAACGCCTACCCCGTGGAG
TGCTGGATCCCCGATCCCTCCCGCAACGAGAATACCCGTTACTTCGGC
ACCTACACCGGTGGCGAGAATGTGCCACCCGTGCTCCACGTTACCAAC
ACCGCCACCACCGTGCTCCTGGATGAGCAGGGAGTCGGCCCACTGTGC
AAGGCTGATAGCCTGTACGTTTCCGCCGCTGACATCTGCGGCCTGTTC
ACTAATTCCTCCGGCACTCAGCAGTGGCGTGGCCTGCCCCGTTACTTC
AAGATCCGTCTCCGTAAGCGTTCCGTGAAGAACCCCTACCCCATCAGC
TTCCTGCTGTCCGACCTCATTAACCGCCGCACCCAGCGTGTGGATGGC
CAGCCCATGTACGGCATGGAGTCCCAGGTGGAGGAGGTTCGTGTCTTC
GACGGCACCGAGCAGTTGCCCGGCGACCCAGACATGATCCGCTATATC
GACCGCCAGGGTCAGCTGCAGACCAAGATGGTC-3′

The encoded amino acid translation (polypeptide) of this nucleotide sequence is:

N-term-MAPTKRKGECPGAAPKKPKEPVQVPKLLIKGGVEVLEVKTG
VDAITEVECFLNPEMGDPDNDLRGYSLRLTAETAFDSDSPDRKMLPCY
STARIPLPNLNEDLTCGNLLMWEAVTVKTEVIGITSMLNLHAGSQKVH
ENGGGKPVQGSNFHFFAVGGDPLEMQGVLMNYRTKYPEGTVTPKNPTA
QSQVMNTDHKAYLDKNNAYPVECWIPDPSRNENTRYFGTYTGGENVPP
VLHVTNTATTVLLDEQGVGPLCKADSLYVSAADICGLFTNSSGTQQWR
GLPRYFKIRLRKRSVKNPYPISFLLSDLINRRTQRVDGQPMYGMESQV
EEVRVFDGTEQLPGDPDMIRYIDRQGQLQTKMV-C-term

The entire open reading frame (ORF) of the codon optimized BK MMR-29 VP1 gene with a Kozak consensus and unique restriction sites at each end (EcoR1/Not1) was artificially engineered by PCR-based gene synthesis (GeneScript, Piscataway, N.J.) and cloned in a pUC18 vector. The modified BK VP1 gene was subcloned between the EcoR1/Not1 sites of the pORB baculovirus transfer vector (Orbigen, San Diego, Calif.). The transfer vector was co-transfected with BaculoGold linear baculovirus DNA (BD Bioscience) in Spodoptera frugiperda sf9 cells using transfection reagents provided in the kit, as suggested by the manufacturer. Seven days post-transfection, the recovered recombinant baculoviruses were further amplified by large scale infections of sf9 cells. Production of VLPs was conducted in Trichoplusia ni (High Five) cells (Invitrogen, Carlsbad, Calif.) infected with an aliquot of the Baculovirus stock. After 96 h of incubation at 27° C., the cells were harvested, and collected by centrifugation at 2,000 rpm (Sorvall FH18/250 rotor) for 15 mM The cell pellet was resuspended in VLP buffer (50 mM Tris pH=7, 1 M NaCl, 2 mM MgCl₂, 1 mM CaCl2), and the VLPs released by 4 freeze-thaw cycles. The lysate was treated for 1 hr at 37° C. with Benzonase (75U/ml) and then clarified by centrifugation at 8,000×g for 30 mM and further dilipidated by Vertrel DF (Fischer) extraction. The aqueous layer from the Vertrel DF extraction was loaded onto a cushion of 40% sucrose in VLP buffer and centrifuged in a SW-41 rotor at 38,000 rpm for 90 mM at 4° C. The resulting pellet was resuspended in VLP buffer, loaded on a discontinuous OptiPrep gradient (26%, 32%), and centrifuged in a SW-41 rotor at 38,000 rpm for 2 h at 16° C. The bands collected at the 26/32 interfaces was collected and diluted 3-fold with VLP buffer, loaded on a discontinuous CsCl gradient (densities of 1.2, 1.3, and 1.4 gr/ml), and centrifuged in a SW-41 rotor at 38,000 rpm for 3 h at 4° C. Capsids were collected from the bottom of the 1.3 phase and stored frozen at −70 C. Purity of the VLP preparation was determined by SDS-PAGE, and capsid formation was verified by electron microscopy. VLP protein was used in an ELISA assay to measure capsid antibodies as performed for BK genotype I and JC viruses (Viscidi et al., 2003; Viscidi et al., 2011). A subset of serum samples was tested in a competitive inhibition (blocking) ELISA assay to determine the specificity of BK 4 seroreactivity. Serum samples were diluted 1:200 in 0.5% (wt vol⁻¹) polyvinyl alcohol (PVA), molecular weight 30,000 to 70,000 (Sigma, St. Louis, Mo.) in Blocker™ casein in PBS (Pierce) containing 0.25 ug/ml of BK4 VLP protein or buffer alone. After incubation for 30 min at 37° C., the serum samples were transferred to a BK4-coated microtiter plate and the ELISA was completed as for the direct ELISA assay. Percent inhibition was calculated as follows: 1 OD value of blocking VLP/OD value of buffer control×100.

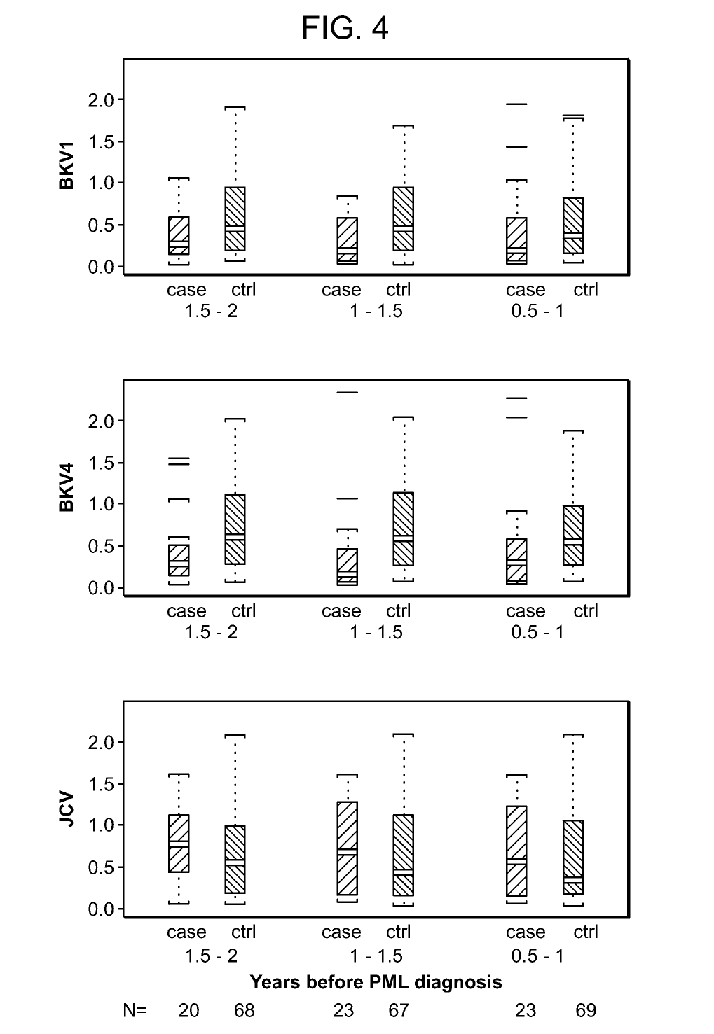

The distribution of IgG seroreactivity to BK genotype I (also designated BK 1), BK genotype IV (also designated BK 4) and JC capsids during 6-month time intervals prior to PML diagnosis is shown in FIG. 4. The association of pre-diagnostic BK and JC serum antibodies levels with risk of PML, stratified by time before PML diagnosis is shown in Table 1.

TABLE 1

| | | | | Unadjusted HR | |
|---|---|---|---|---|---|
| Viral | Yrs from | No. | No. | (confidence | P |
| antibody | Dx | cases | ctrl | interval) | value |
| BK 1 | −0.5, −1 | 23 | 69 | 0.66 (0.45, 0.98) | 0.037 |
| | −1, −1.5 | 23 | 67 | 0.62 (0.44-0.87) | 0.005 |
| | −1.5, −2 | 20 | 68 | 0.73 (0.52-1.03) | 0.073 |
| BK 4 | −0.5, −1 | 23 | 69 | 0.65 (0.44-0.94) | 0.022 |
| | −1, −1.5 | 23 | 67 | 0.50 (0.32-0.78) | 0.002 |
| | −1.5, −2 | 20 | 68 | 0.58 (0.37-0.92) | 0.021 |
| JC | −1, −0.5 | 23 | 69 | 1.20 (0.87-1.65) | 0.27 |
| | −1, −1.5 | 23 | 67 | 1.23 (0.86-1.76) | 0.25 |
| | −1.5, −2 | 20 | 68 | 1.25 (0.87-1.79) | 0.23 |

BK genotype I and BK genotype IV antibody levels were significantly lower in cases compared to controls indicating that BK antibody had a protective effect against the risk of developing PML. In contrast, JC antibody levels were higher among cases than controls, although the difference was not statistically significant. The strength of the association was similar for all three 6-month time intervals, indicating that the relationship between the biomarkers and risk of PML was stable over the 2 year period before PML diagnosis. Because the biomarker was stable over time, data for the three time periods was pooled to increase statistical power. Additionally, BK seroreactivity was expressed as the inverse of the optical density to generate positive associations with the risk of PML, which was the biological outcome of interest. Table 2 shows the unadjusted association of pre-diagnostic BK and JC serum antibody levels during the period between 0.5 and 2 years before diagnosis with the risk of PML (BK serology expressed as inverse level to generate positive associations).

TABLE 2

| Viral | Yrs from | No. case | No. ctrl | | P |
|---|---|---|---|---|---|
| antibody | Dx | specimens | specimens | OR (95% CI) | value |
| Inverse | −0.5, −2 | 66 | 204 | 1.49 (1.11-2.01) | 0.008 |
| BK1 | | | | | |
| Inverse | −0.5, −2 | 66 | 204 | 1.83 (1.29-2.58) | 0.001 |
| BK 4 | | | | | |
| JC | −0.5, −2 | 66 | 204 | 1.16 (0.87-1.55) | 0.32 |

As observed in the analysis of individual time intervals, the analysis of the pooled data show that BK genotype I and IV antibody levels were associated with the risk of PML, with an odds ratio for inverse BK 1 level of 1.49 (p=0.008) and for inverse BK 4 level of 1.83 (p=0.001). Table 3 shows the results of a multivariate (association of 5 pre-diagnostic parameters) logistic model with generalized estimating equations for repeated measures (log 2 inverse BK1, log 2 inverse BK 4, log 2 JC, CD4+ cell count, and age).

TABLE 3

| Obs | Parm¹ | Estimate | Stderr | or | or_lower | or_upper | pvalue |
|---|---|---|---|---|---|---|---|
| 1 | log2_inverse_bkv1_1s | 0.2815 | 0.3163 | 1.33 | 0.71 | 2.46 | 0.373 |
| 2 | log2_inverse_bkv4_1s | 0.7895 | 0.3048 | 2.20 | 1.21 | 4.00 | 0.010 |
| 3 | log2_jcv_1sd | 0.2731 | 0.2589 | 1.31 | 0.79 | 2.18 | 0.292 |
| 4 | cd4cen200_1sd | −0.3888 | 0.2127 | 0.68 | 0.45 | 1.03 | 0.068 |
| 5 | agecen40_1sd | −0.1544 | 0.2626 | 0.86 | 0.51 | 1.43 | 0.557 |

¹To standardize scale for each parameter, measures are expressed as standard deviation.

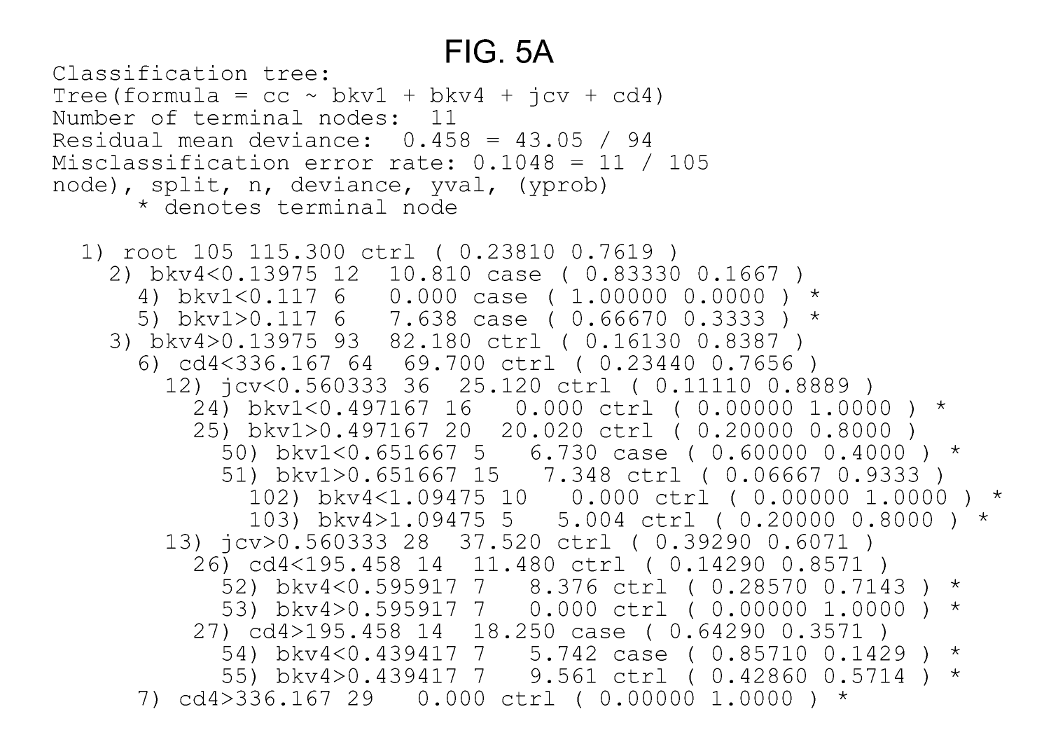

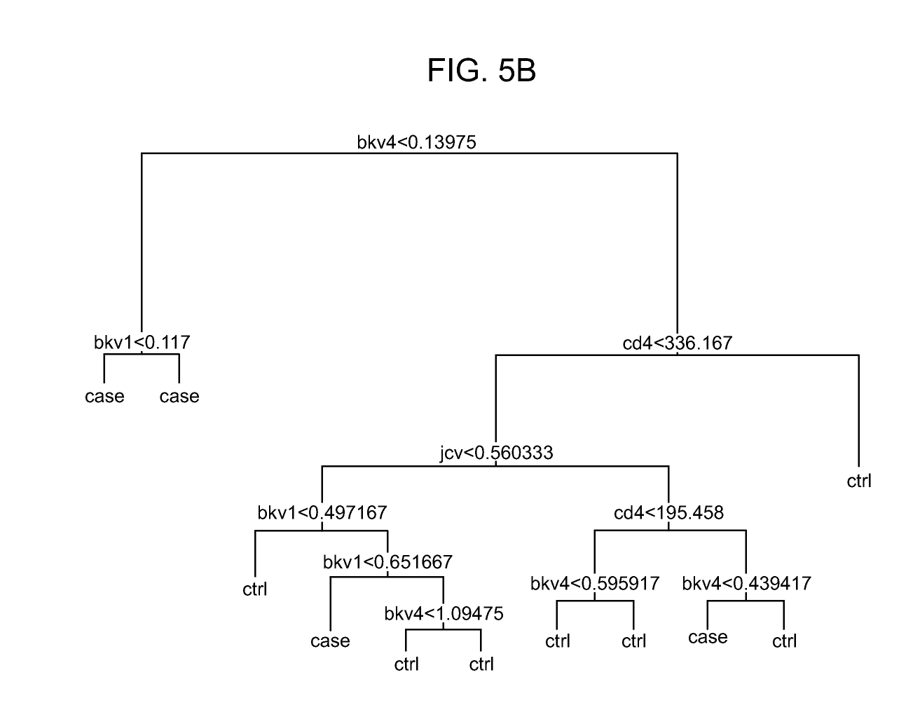

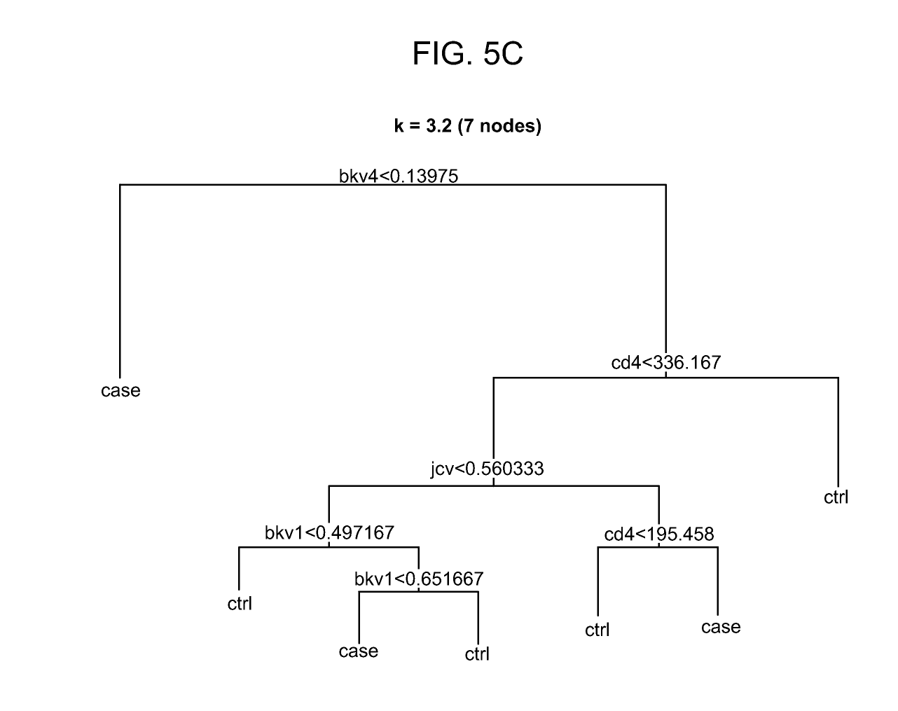

After adjusting for age, CD4+ T cell count, inverse BK genotype I and JCV antibodies, the pre-diagnostic inverse BK genotype IV antibody level was significantly associated with the risk of subsequent PML (odds ratio 2.2, 95% confidence limits, 1.2-4.0; p=0.01). In contrast, and notably for BK genotype I inverse antibody level, none of the other variables after adjustment were significantly associated with risk of PML. A low CD4+ T cell count was associated with the risk of PML but the association did not reach statistical significance (p=0.068). These data provided strong support for the value of BK serology, particularly BK genotype IV seroreactivity, as a pre-diagnostic marker for the risk of development of PML in patients with human immunodeficiency virus infection. To develop an analytical framework that would combine information from the 3 antibody biomarkers and CD4+ T cell count into an algorithm to predict the risk of PML, a decision tree analysis was implemented. In this analysis, data were iteratively stratified into dichotomous categories to achieve optimal differentiation of cases from controls. The analytical results are displayed as a tree-like structure with all data points falling within terminal nodes. For each terminal node, the probability of being a case or control was reported. Ideally, every node would end with a probability of 100% for classification as a case or control. Where probabilities were less than 100%, the node would contain a mixture of cases and controls. The results of the analysis could be expressed as the proportion of data points that were correctly classified. For the decision tree analysis, the average value for each variable was used, so the number of data points corresponded to the number of subjects. FIG. 5A shows the output from the decision tree analysis performed using the variables OD BK genotype I, OD BK genotype IV, OD JCV, and CD4+ T cell count. The 11 terminal nodes from this analysis were indicated with an * and the probabilities were presented in parenthesis (FIG. 5A). The accompanying tree for the analysis that was performed and generated 11 nodes is shown in FIG. 5B. Of the 80 controls, 75 (93.75%) were correctly classified and of the 25 controls, 19 (76%) were correctly classified. The analysis was further developed by pruning the tree, i.e., reducing the number of nodes, in order to optimize the classification of cases and controls. A tree with 7 nodes (FIG. 5C) allowed the correct classification of 89% of controls and 88% of cases. These data and analyses illustrated how certain embodiments of the current invention could be implemented in a particular case to predict the risk of developing PML—here, in HIV patients.

To assess whether genetic variants of the BK genotype IV reagent would enhance the predictive value of a classifier of the invention, a second VLP was produced, composed of a VP1 sequence within the genotype IV family but differing by 4 amino acids from MMR-29. For this purpose, the amino acid sequence of BK IV strain THK8 VP1 protein (Genbank accession number AB211390) was converted in silico to a nucleotide sequence corresponding to the preferred codon usage of Drosophila melanogaster, resulting in the following complete nucleotide sequence:

5′-ATG GCC CCA ACC AAG CGC AAG GGT GAG TGC CCC
GGA GCC GCC CCC AAG AAG CCC AAG GAG CCA GTG CAG
GTC CCC AAG CTG CTG ATC AAG GGT GGA GTT GAG GTG
CTG GAG GTC AAG ACC GGT GTG GAT GCC ATC ACC GAG
GTG GAA TGC TTC CTG AAC CCC GAG ATG GGC GAC CCA
GAT AAC GAT CTG CGC GGC TAT AGC TTG CGC CTG ACC
GCT GAG ACC GCC TTC AAC TCC GAC TCC CCA GAC CGT
AAG ATG CTG CCA TGC TAC TCC ACC GCC CGT ATC CCC
CTG CCC AAC CTG AAC GAG GAC CTG ACC TGC GGA AAC
CTG CTG ATG TGG GAG GCT GTG ACT GTG AAG ACC GAG
GTG ATC GGC ATC ACT AGC ATG CTG AAC CTG CAC GCC
GGC TCC CAG AAG GTT CAC GAC AAC GGA GGC GGC AAG
CCC ATC CAG GGA AGC AAC TTC CAC TTC TTC GCC GTG
GGC GGC GAC CCC CTG GAA ATG CAG GGA GTG CTG ATG
AAC TAC CGC ACC AAG TAC CCC GAG GGC ACC GTC ACC
CCC AAG AAT CCC ACC GCC CAG TCC CAG GTT ATG AAC
ACC GAC CAC AAG GCC TAC TTG GAC AAG AAC AAC GCC
TAC CCG GTC GAG TGC TGG ATT CCC GAC CCA TCC CGT
AAT GAG AAC ACC CGC TAC TTC GGT ACC TAC ACC GGT
GGC GAG AAC GTG CCC CCC GTG CTG CAC GTC ACT AAC
ACC GCC ACC ACC GTG CTG CTG GAT GAA CAG GGT GTC
GGA CCC CTG TGC AAG GCC GAC TCC TTG TAC GTG TCC
GCC GCC GAT ATC TGC GGT CTG TTC ACC AAC TCG TCG
GGT ACC CAG CAG TGG CGT GGC CTG CCA CGC TAC TTC
AAG ATC CGT CTG CGC AAG CGT AGC GTG AAG AAC CCC
TAT CCC ATC TCG TTC CTG CTC TCG GAC CTC ATT AAC
CGT CGT ACC CAG CGT GTG GAT GGA CAG CCA ATG TAC
GGT ATG GAG AGC CAG GTG GAA GAG GTC CGT GTC TTC
GAC GGC ACC GAG AAG CTG CCC GGT GAC CCC GAC ATG
ATC CGC TAC ATC GAT CGC CAG GGC CAG TTG CAG ACC
AAG ATG GTT-3′

The translated amino acid sequence of the above nucleotide sequence differs by 4 amino acids (in bold, underlined 12 point font with space between) from MMR-29:

MAPTKRKGECPGAAPKKPKEPVQVPKLLIKGGVEVLEVKTGVDAITEV
ECFLNPEMGDPDNDLRGYSLRLTAETAF N DSPDRKMLPCYSTARIP
LPNLNEDLTCGNLLMWEAVTVKTEVIGITSMLNLHAGSQKVH D NGG
GKP I QGSNFHFFAVGGDPLEMQGVLMNYRTKYPEGTVTPKNPTAQS
QVMNTDHKAYLDKNNAYPVECWIPDPSRNENTRYFGTYTGGENVPPVL
HVTNTATTVLLDEQGVGPLCKADSLYVSAADICGLFTNSSGTQQWRGL
PRYFKIRLRKRSVKNPYPISFLLSDLINRRTQRVDGQPMYGMESQVEE
VRVFDGTE K LPGDPDMIRYIDRQGQLQTKMV

The ORF of the codon optimized BK THK8 VP1 was artificially engineered by PCR-based gene synthesis as described above for MMR-29. A recombinant baculovirus was constructed and VLPs were purified from insect cells infected with the baculovirus. The VLPs were used in an ELISA assay to measure serum antibodies to THK8 capsids. A subset of the serum samples from the HIV patients (serum obtained from 21 cases and 63 controls in the time window of 1 to 1.5 years prior to PML diagnosis) was tested. The distribution of OD values from cases and controls and the logistic model supported the results obtained using the MMR-29 VLPs, with lower OD values among cases than controls (0.127 vs 0.221) and a statistically significant inverse association between THK8 antibody level and subsequent risk of PML (age and CD4+ T cell count adjusted OR, 0.389, 95% CI, 0.163-0.927, p value=0.033). A decision tree analysis using OD values for BK genotype I, JCV, BK genotype IV (MMR-29) and BK genotype IV (THK8) and CD4+ T cell count correctly classified 81% of cases and 94% of controls as compared to 90% of cases and only 85% of controls for an analysis that did not include THK8. These analyses demonstrated that the association of BK genotype IV seroreactivity with PML was characteristic of the class of genotype IV variants and was not unique to MMR-29. However, the analyses also indicated that additional refinement of risk prediction could be achieved using seroreactivity to both the MMR-29 and THK8 variants of genotype IV BK as classifier inputs, depending upon the goals of the analysis in terms of clinical translation—that is, to maximize classification of cases for optimal sensitivity or of controls for optimal specificity.

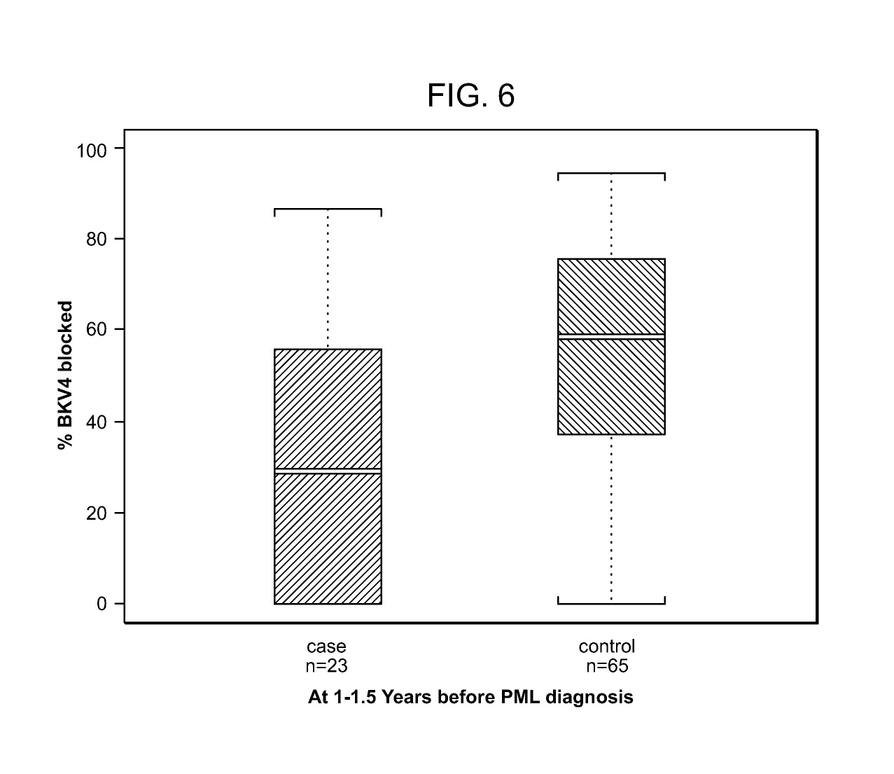

To determine the specificity of reactivity in the BK4 ELISA, a competitive inhibition assay was performed and results were expressed as percent inhibition. Inhibition of BK4 seroreactivity by pre-incubation of serum samples with BK4 VLP protein provided evidence for the specificity of the reactivity, whereas failure to inhibit the reactivity indicated that uncharacterized cross reactive antibodies were responsible for the reactivity Inhibition was a continuous variable but conventionally inhibition greater than 40-50% was interpreted as support for specificity. The observed distribution of BK4 percent inhibition among cases and controls is shown in FIG. 6. Higher percent inhibition (greater BK4 specificity) was characteristic of the seroreactivity of controls compared to cases (median percent inhibition of controls, 59% versus 29% for cases, non-parametric Wilcoxon test p<0.001). These results indicated that BK4 specific seroreactivity was protective against PML. Thus, it was identified that a BK4 competitive inhibition assay could be used in conjunction with assays for BK1, BK4, and JC seroreactivity to improve PML risk prediction.

### Example 3

### Risk Prediction of PML in Patients with Other Indications

For other applications of the invention, the serological markers can be combined with relevant PML-associated risk factors in a decision tree analysis such as those successfully employed above, to derive estimates of the probability of developing PML for a particular risk population. For example, in the case of natalizumab (Tysabri)-associated PML, the number of natalizumab infusions and the use of immunosuppressive therapies prior to initiating natalizumab treatment have been identified to affect the risk of developing PML. BK and JC seroreactivity has been described to vary with age; and thus, in patient populations encompassing a broad age range, age can be included as a variable in the decision tree analysis, to strengthen the analysis. For any such analysis, to optimize the decision tree analysis, it is important to establish the criteria for terminal nodes and to define the probability of PML for each terminal node. In addition, documented cases (patients who developed PML) and controls need to be analyzed; and the accuracy of the risk estimates can be improved by analyzing the greatest available number of cases and controls. Once a decision tree analysis for the risk of PML in a particular patient population has been run, the result can be used to write a software program that will allow clinicians to enter parameters for all the input variables (e.g., measured values) and to calculate a particular patients' risk of developing PML.

### REFERENCES

Berger, J. R. (2010). Progressive multifocal leukoencephalopathy and newer biological agents. Drug Saf 33, 969-983.
Gorelik, L., Lerner, M., Bixler, S., Crossman, M., Schlain, B., Simon, K., Pace, A., Cheung, A., Chen, L. L., Berman, M., Zein, F., Wilson, E., Yednock, T., Sandrock, A., Goelz, S. E., and Subramanyam, M. (2010). Anti-JC virus antibodies: implications for PML risk stratification. Ann. Neurol. 68, 295-303.
Knowles, W. A., Pipkin, P., Andrews, N., Vyse, A., Minor, P., Brown, D. W., and Miller, E. (2003). Population-based study of antibody to the human polyomaviruses BKV and JCV and the simian polyomavirus SV40. J. Med. Virol. 71, 115-123.
Li, J., Melenhorst, J., Hensel, N., Rezvani, K., Sconocchia, G., Kilical, Y., Hou, J., Curfman, B., Major, E., and Barrett, A. J. (2006). T-cell responses to peptide fragments of the BK virus T antigen: implications for cross-reactivity of immune response to JC virus. J. Gen. Virol. 87, 2951-2960.
Major, E. O. (2001). Human Polyomavirus. In Fields Virology, D. M. Knipe and P. M. Howley, eds. (Philadelphia: Lippincott Williams and Wilkins), pp. 2175-2196.
Sharma, M. C., Zhou, W., Martinez, J., Krymskaya, L., Srivastava, T., Haq, W., Diamond, D. J., and Lacey, S. F. (2006). Cross-reactive CTL recognizing two HLA-A*02-restricted epitopes within the BK virus and JC virus VP1 polypeptides are frequent in immunocompetent individuals. Virology 350, 128-136.
Sorensen, P. S., Bertolotto, A., Edan, G., Giovannoni, G., Gold, R., Havrdova, E., Kappos, L., Kieseier, B. C., Montalban, X., and Olsson, T. (2012). Risk stratification for progressive multifocal leukoencephalopathy in patients treated with natalizumab. Mult. Scler. 18, 143-152.
Viscidi, R. P. and Clayman, B. (2006). Serological cross reactivity between polyomavirus capsids. Adv. Exp. Med. Biol. 577, 73-84.
Viscidi, R. P., Rollison, D. E., Sondak, V. K., Silver, B., Messina, J. L., Giuliano, A. R., Fulp, W., Ajidahun, A., and Rivanera, D. (2011). Age-specific seroprevalence of Merkel cell polyomavirus, BK virus, and JC virus. Clin. Vaccine Immunol. 18, 1737-1743.
Viscidi, R. P., Rollison, D. E., Viscidi, E., Clayman, B., Rubalcaba, E., Daniel, R., Major, E. O., and Shah, K. V. (2003). Serological cross-reactivities between antibodies to simian virus 40, BK virus, and JC virus assessed by virus-like-particle-based enzyme immunoassays. Clin. Diagn. Lab Immunol. 10, 278-285.

1. A method for identifying a subject at reduced risk of developing progressive multifocal leukoencephalopathy (PML) due to human immunodeficiency virus-associated immunosuppression or upon administration of a humanized monoclonal antibody targeting immune function comprising detecting serum antibody to BK virus in said subject, wherein the presence of serum antibody to BK virus or a level of antibody to BK virus above a threshold level in said subject identifies a subject at reduced risk of developing PML.

2. The method of claim 1, wherein said method further comprises administering a humanized monoclonal antibody targeting immune function, optionally wherein said humanized monoclonal antibody is selected from the group consisting of natalizumab, rituximab, alemtuzumab and efalizumab.

3. (canceled)

4. A method selected from the group consisting of:
a method of assessing the risk of occurrence of progressive multifocal leukoencephalopathy (PML) in a subject, the method comprising detecting serum antibody to BK virus in a sample from said subject, wherein absence of serum antibody to BK virus or a level of antibody to BK virus below a threshold level indicates an increased risk of occurrence of PML in said subject;
a method of stratifying a subject undergoing α₄-integrin blocking agent treatment and/or VLA-4 blocking agent treatment for suspension of the α₄-integrin and/or VLA-4 blocking agent treatment, the method comprising: (i) detecting serum antibody to JC virus in a sample from said subject and (ii) detecting serum antibody to BK virus in a sample from said subject, wherein: (a) presence of serum antibody to JC virus or an elevated level of serum antibody to JC virus, relative to a threshold level and (b) absence of serum antibody to BK virus or a decreased level of serum antibody to BK virus, relative to a threshold value, indicates that the subject is in need of a suspension of the α₄-integrin-blocking agent treatment and/or VLA-4 blocking agent treatment;
a method for treating or preventing multiple sclerosis (MS), Crohn's or other autoimmune condition in a subject comprising detecting serum antibody to BK virus in a subject and administering a humanized monoclonal antibody targeting immune function to said subject, thereby treating or preventing MS, Crohn's or other autoimmune condition in said subject; and
a method for treating or preventing a neoplastic condition in a subject comprising detecting serum antibody to BK virus in a subject and administering a humanized monoclonal antibody targeting immune function to said subject, thereby treating or preventing said neoplastic condition in said subject.

5. The method of claim 4, wherein detecting the absence of serum antibody to BK virus or level of antibody to BK virus below a threshold level is based on comparison to the level of serum antibody to BK virus in a control sample, optionally wherein said control sample is of individual(s) who developed HIV-associated immunosuppression or who received the monoclonal antibody therapy and were shown not to develop PML.

6. (canceled)

7. The method of claim 1, wherein said subject is undergoing α₄-integrin blocking agent treatment and/or VLA-4 blocking agent treatment optionally wherein said subject has HIV.

8. The method of claim 1, further comprising detecting serum antibody to JC virus in said subject.

9. (canceled)

10. The method of claim 1, wherein a decision tree analysis is performed to predict the risk of PML in the subject, optionally wherein one or more of the following inputs are assessed to predict the risk of PML in the subject: OD BK genotype I, OD BK genotype IV, OD JCV, CD4+ T cell count, BK genotype IV (MMR-29), BK genotype IV (THK8) and BK4 competitive inhibition assay result(s), optionally wherein PML is predicted in the subject with sensitivity selected from the group consisting of at least 80%, at least 85% and at least 90%, optionally wherein PML is predicted in the subject with specificity selected from the group consisting of at least 80%, at least 85% and at least 90%, optionally wherein said subject is being treated for an autoimmune disease or disorder, optionally wherein said autoimmune disease or disorder is selected from the group consisting of multiple sclerosis, Crohn's disease, systemic lupus erythematosus, rheumatoid arthritis, psoriasis, and an idiopathic inflammatory myopathy, optionally wherein the autoimmune disease or disorder is multiple sclerosis or Crohn's disease.

11-17. (canceled)

18. The method of claim 7, wherein the α₄-integrin blocking agent and/or the VLA-4 blocking agent is an immunoglobulin or a proteinaceous binding molecule with immunoglobulin-like functions, optionally wherein said α₄-integrin blocking agent and/or VLA-4 blocking agent is natalizumab.

19-20. (canceled)

21. The method of claim 4, wherein said other autoimmune condition is selected from the group consisting of systemic lupus erythematosus, rheumatoid arthritis, psoriasis, and an idiopathic inflammatory myopathy.

22. (canceled)

23. The method of claim 4, wherein said humanized monoclonal antibody is selected from the group consisting of natalizumab, rituximab, alemtuzumab, and efalizumab.

24. The method of claim 4, wherein said detecting of serum antibody to BK virus involves ELISA.

25. The method of claim 1, wherein said detecting of serum antibody to BK virus comprises differentiating between one or more major genotypes of BK virus selected from the group consisting of I, II, III and IV, optionally wherein said differentiating between one or more major genotypes of BK virus comprises identifying serum antibody specific for a genotype selected from the group consisting of I, II, III and IV, using a method that comprises competitive inhibition of reactivity with soluble antigen of the genotype, optionally wherein said differentiating of major genotypes of BK virus comprises discretely identifying serum antibody to BK virus of a major genotype selected from the group consisting of I and IV, or involves identifying serum antibody to BK virus of only major genotypes I and IV.

26-27. (canceled)

28. The method of claim 1, wherein said detecting is performed upon a sample from said subject.

29. The method of claim 28, wherein said sample is selected from the group consisting of a blood sample and a sample of cerebrospinal fluid.

30. An α₄-integrin and/or VLA-4 blocking agent for use in the treatment of multiple sclerosis (MS), Crohn's, other autoimmune condition or a neoplastic condition so as to avoid the occurrence of PML, wherein the use comprises administering of the α₄-integrin and/or VLA-4 blocking agent to a subject over a period of time, followed by a discontinuation of the administration for a period of time, wherein discontinuation of the administration of the α₄-integrin and/or VLA-4 blocking agent is effected after detecting an absence of serum antibody to BK virus or a decreased level of serum antibody to BK virus relative to a threshold level in said subject.

31. The α₄-integrin and/or VLA-4 blocking agent of claim 30, wherein an absence of serum antibody to BK virus or a decreased level of serum antibody to BK virus relative to a threshold level is detected in a sample from said subject, optionally wherein said sample is selected from the group consisting of a blood sample and a sample of cerebrospinal fluid.

32. (canceled)

33. The α₄-integrin and/or VLA-4 blocking agent of claim 30, wherein serum antibody to JC virus is detected in a sample from said subject.

34. A kit for determining PML risk of a subject comprising an assay for detection of serum antibody to BK virus, and instructions for its use.

35. The kit of claim 34, wherein said assay for detection of serum antibody to BK virus involves ELISA.

36. The kit of claim 34, wherein said kit comprises an assay for detecting one or more of the following: BK genotype I, BK genotype IV, JCV, CD4+ T cell count, BK genotype IV (MMR-29), BK genotype IV (THK8) and BK4 competitive inhibition.
